(19) United States
(12) Patent Application Publication
(10) Pub. No.: US 2019/0390279 A1
(43) Pub. Date: Dec. 26, 2019

(54) METHODS AND KITS FOR USING MTHFR METHYLATION TO CHARACTERIZE THE IMPACT OF TOBACCO USE AND OTHER AGENTS OR CONDITIONS AND/OR TO GAUGE THE INTENSITY OF EXPOSURE TO THE SAME

(71) Applicant: University of Georgia Research Foundation, Inc., Athens, GA (US)

(72) Inventor: Steven R.H. Beach, Statham, GA (US)

(21) Appl. No.: 16/480,880

(22) PCT Filed: Jan. 26, 2018
(86) PCT No.: PCT/US18/15481

§ 371 Date: Jul. 25, 2019

Related U.S. Application Data
(60) Provisional application No. 62/451,180, filed on Jan. 27, 2017.

(51) Int. Cl. C12Q 1/6883

(52) U.S. Cl. CPC C12Q 1/6883; C12Q 2600/154

(57) ABSTRACT

Described herein are methods and kits to detect the methylation state of a CpG dinucleotide in methylene tetrahydrofolate reductase (MTHFR) and uses thereof. In some embodiments the methods can include the steps of providing a biological sample from the subject; contacting DNA from the biological sample with bisulfite under alkaline conditions to produce bisulfite-treated DNA; contacting the bisulfite-treated DNA with a first oligonucleotide probe, wherein the first oligonucleotide probe is complementary to a nucleotide sequence that includes a CpG dinucleotide in the methylene tetrahydrofolate reductase (MTHFR) gene, wherein the first oligonucleotide probe detects either the unmethylated CpG dinucleotide or the methylated CpG dinucleotide; and detecting either the unmethylated CpG dinucleotide or the methylated CpG dinucleotide in the MTHFR gene, wherein methylation of the CpG dinucleotide in the MTHFR gene is associated with amplification of response of the reporter CpG indicating the exposure of the subject to the MMA.

### CROSS-REFERENCE TO RELATED APPLICATIONS

This application claims the benefit of and priority to co-pending U.S. Provisional Patent Application No. 62/451,180, filed on Jan. 27, 2017, entitled “METHODS AND KITS FOR USING MTHFR METHYLATION TO CHARACTERIZE THE IMPACT OF TOBACCO USE AND OTHER AGENTS OR CONDITIONS AND/OR TO GAUGE THE INTENSITY OF EXPOSURE TO THE SAME,” the contents of which is incorporated by reference herein in its entirety.

### STATEMENT REGARDING FEDERALLY SPONSORED RESEARCH OR DEVELOPMENT

This invention was made with government support under grant number 5R01HD030588-16A1 and awarded by the National Institute of Child Health and Human Development (NICHD), grant number R01DA037648 awarded by the National Institute on Drug Abuse (NIDA), and grant number 1P30DA027827 awarded by the National Institute on Drug Abuse. The government has certain rights in the invention.

### SEQUENCE LISTING

This application contains a sequence listing filed in electronic form as an ASCII.txt file entitled 222102-2810_ST25.txt, created on Jan. 26, 2018. The content of the sequence listing is incorporated herein in its entirety.

### BACKGROUND

Self-reported smoking history provides a way of quantifying a particular form of toxic exposure. It is unfortunately quite unreliable, particularly so in key contexts such as underage smoking, smoking among those with co-occurring health problems, and those for whom admission of smoking is stigmatized or associated with negative consequences. Increasing the accuracy of objective approaches to assessment smoking is potentially useful to better identify individuals in these circumstances who are smoking, but may be reticent to self-disclose their smoking status or accurately report the amount of smoking they do. With regard to nascent smoking, accurate objective assessment is likely to be critical for efforts to intervene early, prior to the onset of addiction, when intervention efforts may be more successful. Likewise, increased accuracy of objective evaluation of smoking history and degree of exposure at various levels of cumulative smoking can be beneficial in clinical practice and for clinical research because false or inaccurate reporting of smoking habits can arise for many reasons, resulting in a lack of objective indicators to facilitate diagnosis and treatment. Further, patient history does not shed light on the underlying molecular changes that occur as a result of past and current smoking, and particularly the way these changes may vary due to individual differences.

As such, there exists a focused need for continued improvement in the techniques that allow for objective quantification of cumulative smoking history based on molecular markers of smoke exposure. There is also a need for methods that can enhance prediction of individual differences in longer-term response to smoking at the molecular level. Such solutions can have great potential utility for clinical intervention and for research. Additionally, development of a technology enhancing the capacity to quantify smoking exposure and predict molecular level response to smoking over time illustrates a more general principle of potential use these improved techniques in predicting degree of molecular level response to various methylation modulating agents (MMAs), but are not limited to, a range of potentially useful medical, public health, and health-related applications.

### SUMMARY

Described herein are methods to detect, quantify, or detect and quantify exposure of a subject to methylation modulating agent (MMA) in the subject that can include the steps of providing a biological sample from the subject; contacting DNA from the biological sample with bisulfite under alkaline conditions to produce bisulfite-treated DNA; contacting the bisulfite-treated DNA with a first oligonucleotide probe, wherein the first oligonucleotide probe is complementary to a nucleotide sequence that includes a CpG dinucleotide in the methylene tetrahydrofolate reductase (MTHFR) gene, wherein the first oligonucleotide probe detects either the unmethylated CpG dinucleotide or the methylated CpG dinucleotide; and detecting either the unmethylated CpG dinucleotide or the methylated CpG dinucleotide in the MTHFR gene, wherein methylation of the CpG dinucleotide in the MTHFR gene is associated with amplification of response of the reporter CpG indicating the exposure of the subject to the MMA. The methods can also include the steps of contacting the bisulfite-treated DNA with a second oligonucleotide probe, wherein the second oligonucleotide probe is complementary to a nucleotide sequence that comprises a CpG dinucleotide within the aryl hydrocarbon receptor repressor (AHRR) gene or other reporter nucleotide sequence specific to the MMA being quantified; detecting either the unmethylated CpG dinucleotide or the methylated CpG dinucleotide within the AHRR gene or other reporter nucleotide sequence specific to the MMA being quantified; and conducting a regression analysis using the degree of methylation in the MTHFR gene and the AHRR gene or other reporter nucleotide sequence specific to the MMA being quantified to predict the intensity of the subject's exposure the MMA. The CpG dinucleotide in the MTHFR gene can be selected from the group of: cg01134491, cg01226883, cg02978542, cg05228408, cg05265975, cg08269394, cg08869383, cg10221637, cg11276438, cg12751404, cg14032528, cg14472778, cg17514528, cg17745097, cg18187189, cg21864959, cg22877851, cg23068701, cg23088157, cg23226134, cg23952195, cg25628740, cg27012203, and any combination thereof. In some embodiments, the CpG dinucleotide in the MTHFR gene can be selected from the group of: cg02978542, cg08269394, cg12751404, cg14032528, cg23068701, cg23226134, cg23952195, and any combination thereof. In some embodiments, the CpG dinucleotide within the AHRR gene is selected from the group consisting of: cg05575921, cg21161138, cg26703534, and any combination thereof or other reporter nucleotide sequence specific to the MMA being quantified. The biological sample can be blood or tissue more directly affected by the exposure. In some embodiments, the biological sample cam be a mononuclear cell pellet prepared from the biological sample. The method can further include an amplifying step after the contacting step(s). The methods can further include a sequencing step that can be performed after the amplifying step. The MMA can be selected from the group of: a toxin, a carcinogen, a pharmaceutical compound, a protein, an enzyme, a vitamin, tobacco smoke, a compound found in tobacco smoke, a medical condition, a non-medical condition, and combinations thereof. In some embodiments, the methylation of the CpG dinucleotide in the MTHFR gene can be associated with amplification of response of the reporter CpG and indicates the level of cumulative exposure to the MMA. In some embodiments, the reporter CpG nucleotide can be specific to the MMA. The methods can further include the step of determining the subject's actual exposure to MMA or determining the subject's predicted exposure to the MMA and wherein methylation of the CpG dinucleotide in the MTHFR gene can be associated with amplification of response of the reporter CpG indicating the exposure of the subject to the MMA. The methods can further include the step of predicting the level of genome-wide, organ-wide, or tissue-wide hypomethylation, hypermethylation, or hypermethylation and hypomethylation in the subject. The methods can further include the step of predicting the amplitude of methylation response to exposure of the MMA. The methods can further include the step of predicting disease development in the subject in response to exposure of the MMA. The methods can include the step of prognosing a disease in the subject in response to exposure of the MMA. The method can further include the step of treating the subject for MMA exposure. In some embodiments, the step of treating the subject for MMA exposure can include administering a pharmaceutical to the subject. In some embodiments, step of treating the subject for MMA exposure can include administering behavioral therapy, psychiatric therapy, psychotherapy, a pharmaceutical or a combination thereof to the subject.

Also described herein are kits configured to determine the methylation status of at least one CpG dinucleotide, wherein the kit can include at least one first oligonucleotide probe, wherein the first oligonucleotide probe can be complementary to a nucleotide sequence that includes a CpG dinucleotide in the methylene tetrahydrofolate reductase (MTHFR) gene, wherein the first oligonucleotide probe can detect either the unmethylated CpG dinucleotide or the methylated CpG dinucleotide. The kit can further include a second oligonucleotide probe, wherein the second oligonucleotide probe can be complementary to a nucleotide sequence that includes a CpG dinucleotide within the aryl hydrocarbon receptor repressor (AHRR) gene or other reporter nucleotide sequence specific to the MMA being quantified. In some embodiments, the CpG dinucleotide in the MTHFR gene can be selected from the group of: cg01134491, cg01226883, cg02978542, cg05228408, cg05265975, cg08269394, cg08869383, cg10221637, cg11276438, cg12751404, cg14032528, cg14472778, cg17514528, cg17745097, cg18187189, cg21864959, cg22877851, cg23068701, cg23088157, cg23226134, cg23952195, cg25628740, cg27012203, and any combination thereof. In some embodiments, the CpG dinucleotide in the MTHFR gene can be selected from the group of: cg02978542, cg08269394, cg12751404, cg14032528, cg23068701, cg23226134, cg23952195, and any combination thereof. In some embodiments, the CpG dinucleotide within the AHRR gene can be selected from the group of: cg05575921, cg21161138, cg26703534, and any combination thereof or other reporter nucleotide sequence specific to the MMA being quantified. The kits can include a solid substrate to which the first oligonucleotide probe, the second oligonucleotide probe, or the first oligonucleotide and the second oligonucleotide probe is attached. The substrate is a polymer, glass, semiconductor, paper, metal, gel, hydrogel, or any suitable combination thereof. The substrate can be configured as a microarray or microfluidic chip. In some embodiments, the kit can include a detectable label.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a table demonstrating a correlation matrix for the variables of Example 1.

FIG. 2 shows a table summarizing beta regression models depicting moderating effects of the MTHFR methylation index (mMTHFR) on cg05575921 in response to smoking for sample 1 (AIM).

FIG. 3 shows a table summarizing Beta regression models depicting moderating effects of variation in mMTHFR on cg05575921 in response to smoking for sample 2 (SHAPE).

FIG. 4 shows a table summarizing beta regression models depicting moderating effects of variation at the mMTHFR on cg05575921 in response to smoking for AIM, excluding (n=40) individuals who self-reported no cigarette use across three waves from 17 to 19, but had cotinine value >1.

FIG. 5 shows a table summarizing beta regression models depicting moderating effects of variation at the mMTHFR on cg05575921 in response to smoking for SHAPE, excluding (n=56) individuals who self-reported no cigarette use across three waves from 17 to 19, but had cotinine value >1.

FIG. 6 shows a graph demonstrating explication of the significant interaction effect in FIG. 2 for AIM sample. Beta regression examining effects of cigarette use at ages 17-19 on cg05575921 moderated by mMTHFR (n=293) using all available participants.

FIG. 7 shows a graph demonstrating explication of the significant interaction effect in FIG. 3 for SHAPE sample. Effects of cigarette use at ages 17-19 on cg05575921 moderated by mMTHFR (n=368) using all available participants.

FIG. 8 shows a table summarizing beta regression models depicting the moderating effects of variation at the mMTHFR on cg21161138 for AIM, excluding (n=40) individuals who self-reported no cigarette use across three waves from ages 17 to 19, but had cotinine value >1.

FIG. 9 shows a table summarizing beta regression models depicting the moderating effects of variation at the mMTHFR on cg21161138 for SHAPE, excluding (n=56) individuals who self-reported no cigarette use across three waves from 17 to 19, but had cotinine value >1.

FIG. 10 shows a table summarizing beta regression models depicting the moderating effects of variation at the mMTHFR on cg26703534 in response to smoking for AIM, excluding (n=40) individuals who self-reported no cigarette use across three waves from 17 to 19, but had cotinine value >1.

FIG. 11 shows a table summarizing beta regression models depicting the moderating effects of variation at the mMTHFR on cg26703534 in response to smoking for SHAPE, excluding (n=56) individuals who self-reported no cigarette use across three waves from 17 to 19, but had cotinine value >1.

FIG. 12 shows a table summarizing beta regression models depicting the moderating effects of variation at the mMTHFR24 (using all 24 loci on MTHFR) on cg05575921 in response to smoking for AIM, excluding (n=40) individuals who self-reported no cigarette use across three waves from 17 to 19, but had cotinine value >1. Interaction explicated in FIG. 22.

FIG. 13 shows a table summarizing beta regression models depicting the moderating effects of variation at the mMTHFR24 (using all 24 loci on MTHFR) on cg05575921 in response to smoking, excluding (n=56) individuals who self-reported no cigarette use across three waves from 17 to 19, but had cotinine value >1. Interaction explicated in FIG. 23.

FIG. 14 shows a table demonstrating CpG sites on the MTHFR gene.

FIG. 15 shows a table summarizing beta regression models depicting moderating effects of variation at the mMTHFR18 (using 18 loci on MTHFR after excluding loci associated with annotated SNPs) on cg05575921 in response to smoking for AIM, excluding (n=40) individuals who self-reported no cigarette use across three waves from 17 to 19, but had cotinine value >1. Interaction explicated in FIG. 22.

FIG. 16 shows a table summarizing beta regression models depicting moderating effects of variation at the mMTHFR18 (using 18 loci on MTHFR after excluding annotated SNPs) on cg05575921 in response to smoking for SHAPE, excluding (n=56) individuals who self-reported no cigarette use across three waves from 17 to 19, but had cotinine value >1. Interaction explicated in FIG. 23.

FIG. 17 shows a graph demonstrating the explication of the interaction from FIG. 4 for AIM sample. Effects of cigarette use at ages 17-19 on cg05575921 moderated by mMTHFR (n=253) using AIM data. Excluding those reporting no cigarette use, but with cotinine >1, indicating potential false under reporting or recent onset.

FIG. 18 shows a graph demonstrating the explication of the interaction effect for FIG. 5 for SHAPE sample. Effects of cigarette use at ages 17-19 on cg05575921 moderated by mMTHFR (n=321) using SHAPE data. Excluding those reporting no cigarette use, but with cotinine >1, indicating potential false under reporting or recent onset.

FIG. 19 shows a graph demonstrating the explication of the interaction effect in FIG. 8. Effects of cigarette use at ages 17-19 on cg21161138 moderated by mMTHFR (n=253) using AIM data. Excluding those reporting no cigarette use, but with cotinine >1, indicating potential false under reporting or recent onset.

FIG. 20 shows a graph demonstrating the explication of the interaction effect in FIG. 9. Effects of cigarette use at ages 17-19 on cg21161138 moderated by mMTHFR (n=321) using SHAPE data. Excluding those reporting no cigarette use, but with cotinine >1, indicating potential false under reporting or recent onset.

FIG. 21 shows a graph demonstrating the explication of the interaction effect in FIG. 10. Effects of cigarette use at ages 17-19 on cg26703534 moderated by mMTHFR (n=253) using AIM data. Excluding those reporting no cigarette use, but with cotinine >1, indicating potential false under reporting or recent onset.

FIG. 22 shows a graph demonstrating the explication of the interaction from FIG. 12 of cigarette use at ages 17-19 and mMTHFR24 computed as mean of all 24 MTHFR* associated loci on cg05575921 for the AIM data set (n=253). Those with potential false under reporting or recent onset indicated by self-report of no cigarette use, but with cotinine >1 were excluded. *mMTHFR24 computed as mean of all MTHFR associated loci. [mMTHFR24=mean (zcg01134491, zcg01226883, zcg02978542, zcg05228408, zcg05265975, zcg08269394, zcg08869383, zcg10221637, zcg11276438, zcg12751404, zcg14032528, zcg14472778, zcg17514528, zcg17745097, zcg18187189, zcg18276943, zcg21864959, zcg22877851, zcg23068701, zcg23088157, zcg23226134, zcg23952195, zcg25628740, zcg27012203).

FIG. 23 shows a graph demonstrating the interaction from FIG. 13 of cigarette use at ages 17-19 and mMTHFR24 computed as mean of all 24 MTHFR* associated loci on cg05575921 for the SHAPE data set (n=321). Those with potential false under reporting or recent onset indicated by self-report of no cigarette use, but with cotinine >1 were excluded. *mMTHFR24 computed as mean of all MTHFR associated loci. [mMTHFR24=mean (zcg01134491, zcg01226883, zcg02978542, zcg05228408, zcg05265975, zcg08269394, zcg08869383, zcg10221637, zcg11276438, zcg12751404, zcg14032528, zcg14472778, zcg17514528, zcg17745097, zcg18187189, zcg18276943, zcg21864959, zcg22877851, zcg23068701, zcg23088157, zcg23226134, zcg23952195, zcg25628740, zcg27012203).

FIG. 24 shows a graph demonstrating the interaction from FIG. 15 of cigarette use at ages 17-19 and mMTHFR18 computed as mean of all 18 MTHFR* associated loci on cg05575921 for the AIM data set (n=253). Those with potential false under reporting or recent onset indicated by self-report of no cigarette use, but with cotinine >1 were excluded. *mMTHFR18 computed as mean of all MTHFR associated loci. [mMTHFR18=mean (zcg01134491, zcg01226883, zcg02978542, zcg08869383, zcg10221637, zcg11276438, zcg12751404, zcg14032528, zcg14472778, zcg17745097, zcg18187189, zcg22877851, zcg23068701, zcg23088157, zcg23226134, zcg23952195, zcg25628740, zcg27012203).

FIG. 25 shows a graph demonstrating the interaction from FIG. 16 of cigarette use at ages 17-19 and mMTHFR18 computed as mean of all 18 MTHFR* associated loci on cg05575921 for the SHAPE data set (n=321). Those with potential false under reporting or recent onset indicated by self-report of no cigarette use, but with cotinine >1 were excluded.

FIG. 26 shows a table demonstrating the moderating effect of variation of mMTHFR on cg05575921 in response to cigarette use for the FACHS middle-aged sample. Beta regression models are utilized. The interaction is explicated in FIG. 27.

FIG. 27 shows a figure demonstrating the explication of the significant interaction effect in FIG. 26 for the FACHS middle-age sample. Beta regressions were used to examine the effect of cigarette use at Waves 3-5 on cg05575921 moderated by mMTHFR (n=180).

FIG. 28A shows a table summarizing significant interaction effects between smoking and mMTHFR in a middle aged sample, showing that MTHFR exaggerates methylation remodeling in response to smoking.

FIG. 28B shows a graph demonstrating the shape of the interaction effects summarized in the first row of FIG. 28, i.e., for those loci showing long-term hypo methylation in response to smoking and that are influenced by variation at mMTHFR. The contrast of those 1 sd below the mean on mMTHFR with those 1 sd above the mean on mMTHFR Indicates that there is more exaggerated long-term remodeling in response to smoking among those with lower mMTHFR

FIG. 28C shows a graph demonstrating the shape of the interaction effects for those loci showing long-term hyper methylation in response to smoking and that are influenced by variation at mMTHFR. The contrast of those 1 sd below the mean on mMTHFR with those 1 sd above the mean on mMTHFR indicates that there is more exaggerated long-term remodeling in response to smoking among those with lower mMTHFR.

FIG. 29 shows a table demonstrating loci that are significant genomewide reporters of level of exposure to air toxins with carcinogenic potential according to the EPA's national-scale Air Toxic Assessment (NATA). Two loci were significantly associated after genome wide corrections: cg16925090 and cg26725838

FIG. 30 shows a table demonstrating regression models indicating that variation in a methylation index of the loci shown to be responsive to air toxin exposure in FIG. 29 (i.e., an index formed by taking the mean of (Zcg16925090 and Zcg26725838), is influenced by MTHFR and that variation in mMTHFR moderates the impact of NATA-based cancer-related toxin exposure due to air quality on the level of the exposure index.

FIG. 31 shows a table demonstrating that gene expression of MTHFR provides an alternative reporter of mMTHFR effects, showing the mirror image moderating effect of MTHFR on the smoking-CG055 relationship using MTHFR expression in interaction with smoking.

FIG. 32 shows a table demonstrating that the effects observed in FIG. 31 are also present after excluding potential false positives. Beta regression models depict the moderating effects of variation in MTHFR expression on cg05575921 in response to smoking, excluding (n=64) individuals who self-reported no cigarette use past eight years, but had cotinine value >1.

FIG. 33 shows a figure illustrating the mirror image moderating effect of MTHFR expression on cg05575921 in response to smoking that was demonstrated in FIG. 32. Effects of cigarette use at ages 21-29 on cg05575921 moderated by variation in MTHFR expression (n=334) using all available participants.

FIG. 34 shows the location of the MTHFR gene which is located at base pairs 11,785,730 to 11,806,103 on chromosome 1 (Homo sapiens Annotation Release 108, GRCh38.p7). Credit: genome decoration page/NCBI.

FIG. 35 shows portions of the body that can be affected by smoking.

FIG. 36 shows base pair sequence data for the region around the first exon of MTHFR (SEQ ID NO: 1), with seven key CG sites underlined and numbered from 01 to 07, corresponding to Illumina array sites 13-19 (cg02978542, cg08269394, cg12751404, cg14032528, cg23068701, cg23226134, cg23952195). The sequence was obtained from the UCSC Genome Browser, available online. All numbered sites are covered by the Illumina Human Methylation 450K Microarray chip.

FIG. 37 shows SEQ ID NO: 2, which demonstrates base pair sequence data for AHRR, CG sites bolded and underlined, with the key reporter site (CG05575921 highlighted in and underlined. The range of the base pairs on Chromosome 5 is 372818-373939.

FIG. 38 shows the target region for cg14032528.

FIG. 39 shows the bisulfite conversion of the target region shown in FIG. 38.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genomics, genetics, molecular biology, microbiology, nanotechnology, organic chemistry, biochemistry, botany and the like, which are within the skill of the art. Such techniques are explained fully in the literature.

### Definitions

As used herein, “about,” “approximately,” and the like, when used in connection with a numerical variable—generally refers to the value of the variable and to all values of the variable that are within the experimental error (e.g., within the 95% confidence interval for the mean) or within +/−10% of the indicated value, whichever is greater.

As used herein, “antibody” can refer to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein, “cDNA” can refer to a DNA sequence that is complementary to a RNA transcript in a cell. It is a man-made molecule. Typically, cDNA is made in vitro by an enzyme called reverse-transcriptase using RNA transcripts as templates.

As used herein, “control” can refer to an alternative subject or sample used in an experiment for comparison purpose and included to minimize or distinguish the effect of variables other than an independent variable.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “DNA molecule” can include nucleic acids/polynucleotides that are made of DNA.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, the term “encode” can refer to principle that DNA can be transcribed into RNA, which can then be translated into amino acid sequences that can form proteins.

As used herein, “gene” can refer to a hereditary unit corresponding to a sequence of DNA that occupies a specific location on a chromosome and that contains the genetic instruction for a characteristic(s) or trait(s) in an organism. The term gene can refer to both translated and untranslated regions of a subject's genome.

As used herein, “identity,” is a relationship between two or more nucleotide or polypeptide sequences, as determined by comparing the sequences. In the art, “identity” also refers to the degree of sequence relatedness between nucleotide or polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides of the present disclosure, unless stated otherwise.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “mammal,” for the purposes of treatments, can refer to any animal classified as a mammal, including human, domestic and farm animals, nonhuman primates, and zoo, sports, or pet animals, such as, but not limited to, dogs, horses, cats, and cows.

The term “molecular weight”, as used herein, can generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “nucleic acid sequence” and “oligonucleotide” also encompasses a nucleic acid and polynucleotide as defined above. As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof.

As used herein “peptide” refers to chains of at least 2 amino acids that are short, relative to a protein or polypeptide.

As used herein, “positive control” can refer to a “control” that is designed to produce the desired result, provided that all reagents are functioning properly and that the experiment is properly conducted.

As used herein, “protein” as used herein can refer to a molecule composed of one or more chains of amino acids in a specific order. The term protein is used interchangeable with “polypeptide.” The order is determined by the base sequence of nucleotides in the gene coding for the protein. Proteins are required for the structure, function, and regulation of the body's cells, tissues, and organs.

As used herein, “purified” or “purify” can be used in reference to a nucleic acid sequence, peptide, or polypeptide that has increased purity relative to the natural environment.

As used interchangeably herein, “subject,” “individual,” or “patient” can refer to a vertebrate organism, such as a mammal (e.g. human).

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used herein, “substrate” can refer to any solid support to which the probes provided herein can be attached. The substrate can be modified, covalently or otherwise, with coating(s) and/or functional groups that can facilitate, inter alia, attachment of the probes. Suitable substrate materials include, but are not limited to, polymers, glasses, semiconductors, papers, metals, gels, hydrogels, and any combinations thereof. The substrate can have any physical shape or size, e.g. plates, strips, microparticles, channels, chips, etc.

As used herein “attached” as applied to probes of an array can refer to a covalent interaction or other bond between a surface of the substrate and the probe so as to immobilize the probe on the surface of the substrate.

As used herein “essentially discrete” as applied to features of an array refers to the situation where 90% or more of the features of an array are not in direct contact with other features of the same array.

As used herein “CpG” can refer to a Cysteine (“C”) and guanine (“G”) nucleotides that are connected by a phosphodiester bond and can refer to these specific CG sequences located in a CpG island.

As used herein “CpG island” can refer to short stretches of DNA where the frequency of CpG dinucleotide sequence is greater relative to other regions of the DNA. Commonly, methylation (and demethylation) is more dynamic at these CpG islands relative to other regions of the DNA.

Unless otherwise defined herein, all technical and scientific terms used herein have the same meaning as commonly understood by one of ordinary skill in the art.

Discussion

Smoking has been to shown to have a well-defined epigenetic effect on a user's DNA. Specifically, smoking can alter the methylation status of DNA, which in turn may alter the transcription of particular genes. Indeed, an early effect of smoking is demethylation of AHRR, particularly at cg05575921 and attempts have been made to use level of methylation of cg05575921 in AHRR (see e.g. U.S. Pat. Nos. 8,637,652 and 9,273,358) to determine a subject's tobacco use. However, these attempts are limited because they do not capture individual differences in methylation response to smoking. Individual differences may arise due to possible underlying variances in other molecular processes involved in methylation of DNA. For example, the availability of methyl donors may influence the degree of demethylation. Accounting for such individual differences can allow for improved methods for quantifying the degree demethylation or hypomethylation resulting from cigarette smoking and may also provide enhanced quantification of a subject's cumulative tobacco use, particularly nascent use, for which self-reporting is most problematic.

Indeed, while self-reported smoking history provides a way of quantifying a particular form of toxic exposure, it is unfortunately quite unreliable, particularly so in key contexts such as underage smoking, smoking among those with co-occurring health problems, and those for whom admission of smoking is stigmatized or associated with negative consequences. Increasing the accuracy of objective approaches to assessment of smoking is potentially useful to improve identification of individuals who are smoking and who may also be reticent to self-disclose their smoking status or accurately report the amount of smoking they do. With regard to nascent smoking, accurate objective assessment is important for efforts to intervene early, prior to the onset of addiction, when intervention efforts may be more successful. Likewise, increased accuracy of objective evaluation of smoking history and degree of exposure at various levels of cumulative smoking can be beneficial in clinical practice and for clinical research because false or inaccurate reporting of smoking habits or exposure can result in a lack of objective indicators to facilitate diagnosis and treatment. Patient history fails to shed light on the underlying molecular changes that occur as a result of past and current smoking or exposure, and particularly fails to reveal the way these changes may vary between individuals.

Methylene tetrahydrofolate reductase (MTHFR) is the rate-limiting enzyme in the methyl cycle, a pathway that is important to folate metabolism, and so may be a source of individual differences in response to stimuli that perturb or alter methylation. Because MTHFR lies at the intersection of pathways for methylation and DNA synthesis it may exert regulatory influence on response to demethylating and hypermethylating agents and conditions. Quantifying methylation of loci on the MTHFR gene (mMTHFR) is of interest due to its potential to afford more accurate quantification of degree of exposure to demethylating and hypermethylating agents by better accounting for effects due to individual differences in key regulatory pathways, or as a tool to enhance prediction of subsequent sequelae that are due, in part, to variation in methylation or patterns of methylation.

With that said, described herein are methods for detecting that a subject is likely to show a greater or lesser response to a particular MMA; methods to quantify individual differences in methylation response to a particular MMA; and a method to enhance determination of exposure intensity based on detection of methylation of one or more CpG dinucleotides in the methylene tetrahydrofolate reductase (MTHFR) gene, in conjunction with detection of methylation of one or more CpG dinucleotides that serve as a reporter for the given MMA. In the case of nascent cigarette smoking key reporter CpGs are in AHRR. For more established smokers, the number of potential reporter CpG sites can be increased. Similarly, the reporter CpG sites for various MMAs, will vary depending on the specific locus of action for each MMA. For the case of nascent smoking, the degree of methylation of MTHFR and AHRR can be used to better predict a subject's demethylation in response to tobacco use and/or to better quantify the intensity of exposure in the absence of self-report.

Assessment of methylation of MTHFR (mMTHFR), can allow for objective quantification and analysis of smoking history in a subject and can enhance prediction of individual differences in response to smoking (including longer-term responses) at the molecular level. Thus, assessment of methylation of MTHFR has potential for use in clinical intervention and treatment of smoking and exposure to MMAs. Moreover, development of a technology that can enhance the capacity to quantify smoking exposure or other MMAs and predict molecular level response to smoking over time can illustrate the more general principle of the use of mMTHFR in predicting the degree of molecular level response to one or more MMAs, which can include a range of potentially useful medical, public health, and health-related applications for the assessment of mMTHFR.

Further, efforts to better quantify degree of smoking and degree of demethylation resulting from nascent smoking illustrate the broad utility of the methods being proposed. Indeed, many of the embodiments and examples focus on smoking because of the strong research tradition that has arisen around smoking's robust and statistically significant demethylating effect on specific molecular markers, providing a strong test of the role mMTHFR plays in moderating the impact of smoking on methylation and demonstrating its potential practical utility. Specifically, methods useful in predicting degree of methylation at key reporter sites in response to smoking (e.g. cg05575921) can be useful in quantifying degree of methylation at key reporter sites for other MMAs.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Methods for Detecting Level of Exposure to a MMA

Methylene tetrahydrofolate reductase (MTHFR) is the rate-limiting enzyme in the methyl cycle, a pathway that is important to folate metabolism, and so is a likely source of individual differences in response to stimuli that perturb or alter methylation. Smooth functioning of this cycle maintains adequate levels of S-adenosylmethionine (SAM) in cells, providing a readily available methyl group donor for numerous methylation related reactions (Stover, 2009), especially genomic methylation. Because MTHFR lies at the intersection of pathways for methylation and DNA synthesis it has unique potential to exert regulatory influence on response to demethylating and hypermethylating agents and conditions. Methylation of MTHFR may be of interest for better quantifying degree of exposure by better accounting for effects due to individual differences in key regulatory pathways, as in the current example, or it may be of interest as a tool in predicting subsequent sequelae that are due, in part, to variation in methylation or patterns of methylation.

With that in mind, provided herein are methods to detect and/or quantify exposure to a MMA, in a subject that can include the steps of providing a biological sample from the subject, contacting DNA from the biological sample with bisulfite under alkaline conditions to produce bisulfite-treated DNA, contacting the bisulfite-treated DNA with a first oligonucleotide probe, wherein the first oligonucleotide probe is complementary to a nucleotide sequence that comprises a CpG dinucleotide in the methylene tetrahydrofolate reductase (MTHFR) gene, wherein the first oligonucleotide probe detects either the unmethylated CpG dinucleotide or the methylated CpG dinucleotide, and detecting either the unmethylated CpG dinucleotide or the methylated CpG dinucleotide in the MTHFR gene, wherein methylation of the CpG dinucleotide in the MTHFR gene is associated with amplification of response of the reporter CpG indicating the exposure of the subject to the MMA. In some embodiments, the methylation of the CpG dinucleotide in the MTHFR gene is associated with amplification of response of the reporter CpG indicating the subject's use of, or intensity thereof, tobacco. It will be understood that the term “probe” herein can refer to nucleotide sequences (such as oligonucleotides, which can be nucleic acid primers or primer pairs (which includes both RNA and DNA primers) and other molecules, such as antibodies and aptamers. Bisulfite treatment of DNA can be used to deaminate unmethylated cytosine to produce uracil in DNA. Upon sequencing and/or amplification using a specific probe and/or primers, the methylation status of the DNA can be detected based on identification of a change in base from cytosine to uracil. The method can also be used to detect that a subject is a tobacco user and/or the intensity of tobacco use by the subject.

The method can further include the steps of contacting the bisulfite-treated DNA with a second oligonucleotide probe, wherein the second oligonucleotide probe is complementary to a nucleotide sequence that comprises a CpG dinucleotide within the reporter gene, in the case of cigarette smoking this can be one or more CpG dinucleotides within the aryl hydrocarbon receptor repressor (AHRR) gene, detecting either the unmethylated CpG dinucleotide or the methylated CpG dinucleotide within the reporter gene, and conducting a moderated regression analysis using the degree of methylation in the MTHFR gene and the reporter gene to identify the extent to which the impact of the subject's exposure to the MMA, on the CpG within the reporter gene is different depending on level of methylation in the MTHFR gene. In some embodiments, methylation in the MTHFR gene and the AHRR gene are used to assess the intensity of the subject's smoking or tobacco product use, or exposure to one or more MMAs in a tobacco product and/or tobacco smoke, and/or their time of cessation.

The method can further include comparing the regression analysis results with a threshold regression coefficient value and/or reference regression coefficient that was obtained from a regression analysis performed on a reference subject or population. The reference subject or population in the case of cigarette smoking can be non-smoker(s), known smoker(s), or other reference person or population. In the case of detection of response to other exposures the reference subject of population can be those known to be not exposed or other reference population. For the example of cigarette smoking, the regression analysis between methylation MTHFR and AHRR can indicate or detect the intensity of smoking (i.e. how much the subject has smoked and/or how long ago the subject ceased smoking). In some embodiments, the regression analysis can be performed using a methylation index (m) for MTHFR and/or AHRR. The methylation index can be derived using the methylation data from two or more CpG dinucleotides or loci for each MTHFR and/or AHRR. In some embodiments, the mMTHFR can be generated from the data from each of the following loci: cg02978542, cg08269394, cg12751404, cg14032528, cg23068701, cg23226134, cg23952195 in MTHFR.

More generally, by examining the impact of exposure to a MMA on a reporter CpG site in a representative sample, the ability to predict response of the reporter CpG site(s) to exposure, both rapid and longer-term, can be enhanced. In some embodiments, the level of methylation at the reporter CpG site can be used in conjunction with methylation of MTHFR to provide an enhanced quantitative index of MMA exposure. Using standard statistical approaches known well to those who work in the area, standardized weights based on mMTHFR can be constructed with which to multiply the value of the reporter loci. In other embodiments, standard reference curves can be constructed relating methylation of the reporter CpG to exposure in a manner that varies as a function of mMTHFR. Generally, toxin or other demethylating and/or hypermethylating agent exposure (e.g. cigarette exposure, smog exposure, carcinogen exposure, MMA etc.) can be calculated as: exposure=b0+b1 (methylation of reporter loci, e.g. cg05575921)+e; with b1 values varying depending on measured level of mMTHFR. No limitation on statistical models or underlying distributional assumptions to be utilized is implied or intended. Similarly, when exposure is known or can be assessed independently, mMTHFR level can be used to predict likely impact on loci that are hypo or hyper methylated in response to the exposure, providing information about systemic impact.

As used herein, MMA can refer to any compound(s) and/or condition(s) (physical, environmental, behavioral, neurological, or otherwise) that can alter the methylation status at one or more CpG sites in the genome. In some aspects, the MMA can cause hypermethylation at one or more CpG site. In some aspects the MMA can cause hypomethylation at one more CpG sites. In some aspects, the MMA can cause hypermethylation at one or more CpG sites in the MTHFR gene, the AHRR gene, or the MTHFR and the AHRR genes. In some aspects, the MMA can cause hypomethylation at one or more CpG sites in the MTHFR gene, the AHRR gene, or the MTHFR and the AHRR genes. The MMAs can be any environmental toxin, medication, compound, medical condition, environmental condition, enzyme or activity thereof found to be statistically significantly associated with changes in methylation at one or more CpG sites, using standard statistical tests and methods well known to those skilled in the art. In some embodiments, the agent can be a compound in smoke from a cigarette. The MMA can be carcinogenic or non-carcinogenic. The MMA can be organic or inorganic. The MMA can be a protein. The MMA can be a microorganism. The MMA can be a heavy metal. The MMA can be any compound including, but not limited, to chemical compounds, biological compounds (including, but not limited to, proteins (including, but not limited to, enzymes) and polynucleotides), nutrients (including but not limited to vitamins (including, but not limited to, folate), fats, fatty acids, amino acids, and minerals), therapeutic and/or preventive pharmaceutical compounds (including but not limited to small molecule drugs, liposomal formulations, etc.). The enzyme can be any enzyme, including, but not limited to, those involved in methylating and/or demethylating DNA.

The method can further include the step of determining the subject's actual exposure to the MMA or determining the subject's predicted exposure to the MMA and wherein methylation of the CpG dinucleotide in the MTHFR gene can be associated with amplification of response of the reporter CpG indicating the exposure of the subject to the MMA. The step of determining the actual exposure in the subject in response to exposure of the MMA can be conducted by determining and/or quantifying the methylation status of MTHFR at one or more CpG dinucleotides and/or methylation status of the AHRR at one or more CpG dinucleotides as described herein, which can be determined by comparing the MTHFR CpG dinucleotide profile as described herein to appropriate controls. Appropriate controls will be able to be determined by one of ordinary skill in the art.

The method can further include the step of predicting the amplitude of methylation response to exposure of the MMA. The method can further include the step of predicting disease or side-effect development in the subject in response to exposure of the MMA. The method can further include the step of prognosing a disease in the subject in response to exposure of the MMA. The step of predicting or prognosing a disease in the subject in response to exposure of the MMA can be conducted by determining and/or quantifying the methylation status of MTHFR at one or more CpG dinucleotides and/or methylation status of the AHRR (for smoking, or other CpG dinucleotides statistically associated for particular MMAs) at one or more CpG dinucleotides as described herein, which can be determined by comparing the MTHFR CpG dinucleotide profile as described herein to appropriate controls. Appropriate controls will be able to be determined by one of ordinary skill in the art.

The reporter CpG dinucleotide can be specific to the MMA. In other words, a MMA can have one or more CpG dinucleotide that is associated with exposure of the subject to the MMA, which can be determined by comparing the MTHFR CpG dinucleotide profile as described herein to appropriate controls. Appropriate controls will be able to be determined by one of ordinary skill in the art.

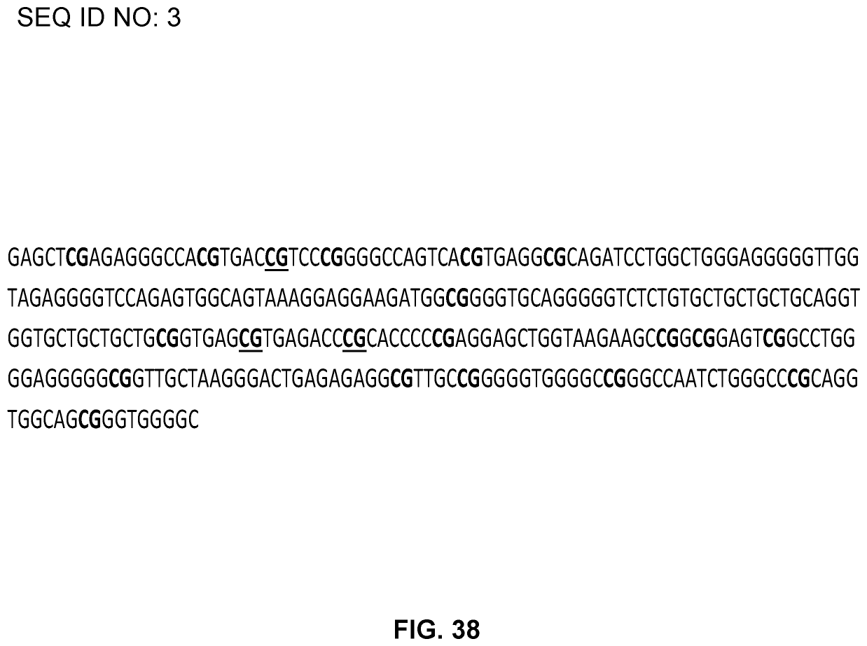

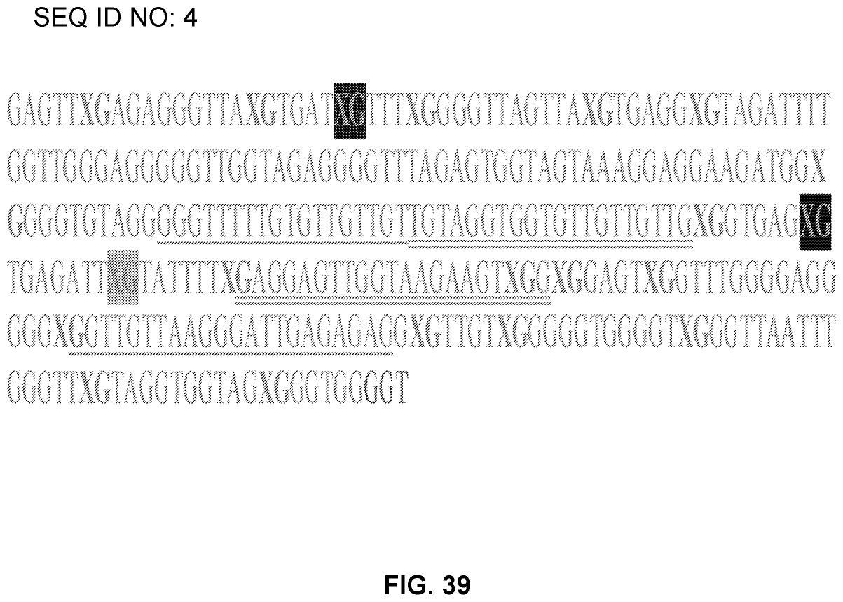

The CpG dinucleotide in the MTHFR gene can be selected from the group of: cg01134491, cg01226883, cg02978542, cg05228408, cg05265975, cg08269394, cg08869383, cg10221637, cg11276438, cg12751404, cg14032528, cg14472778, cg17514528, cg17745097, cg18187189, cg21864959, cg22877851, cg23068701, cg23088157, cg23226134, cg23952195, cg25628740, cg27012203, or other CpG loci in the MTHFR gene and any combination thereof. In some embodiments, the CpG dinucleotide in the MTHFR gene can be selected from the group consisting of: cg02978542, cg08269394, cg12751404, cg14032528, cg23068701, cg23226134, cg23952195, or any combination thereof. In some embodiments, the CpG dinucleotide can be cg14032528. In some embodiments, the target region for cg14032528 can be as shown in FIG. 38. FIG. 39 shows the bisulfite conversion of the target region shown in FIG. 38.

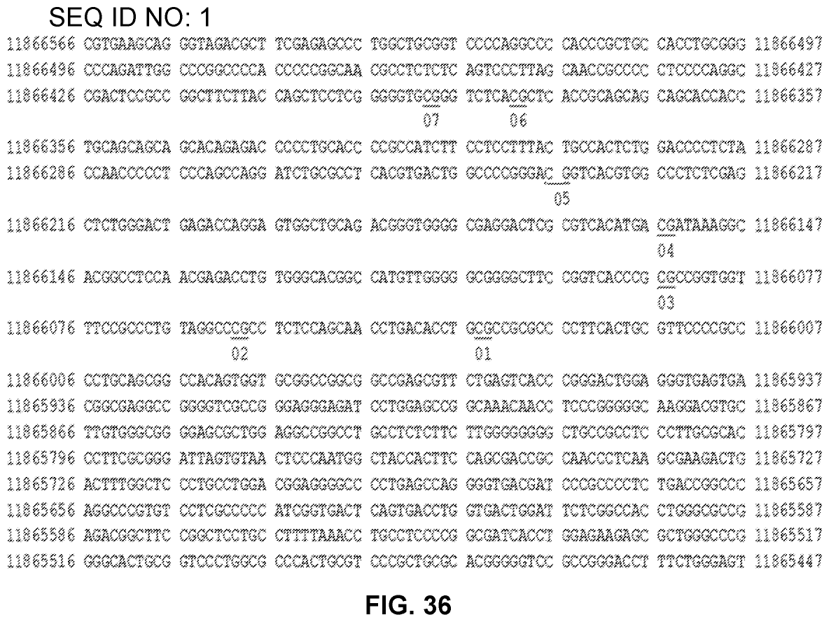

As can be seen in FIG. 36, the base pair sequence provides sufficient information to allow someone skilled in the art to create primers to identify methylation levels at any of the individual CpG sites for MTHFR identified above. The sequence in FIG. 36 was obtained from the UCSC Genome Browser (available online). The seven CpG sites from exon 1 referenced above are underlined and numbered 1-7. Because all are sites covered by the Illumina Human Methylation 450K Microarray chip, they also can also be labeled with the numbers used by Illumina (e.g., No. 13 to No. 19).

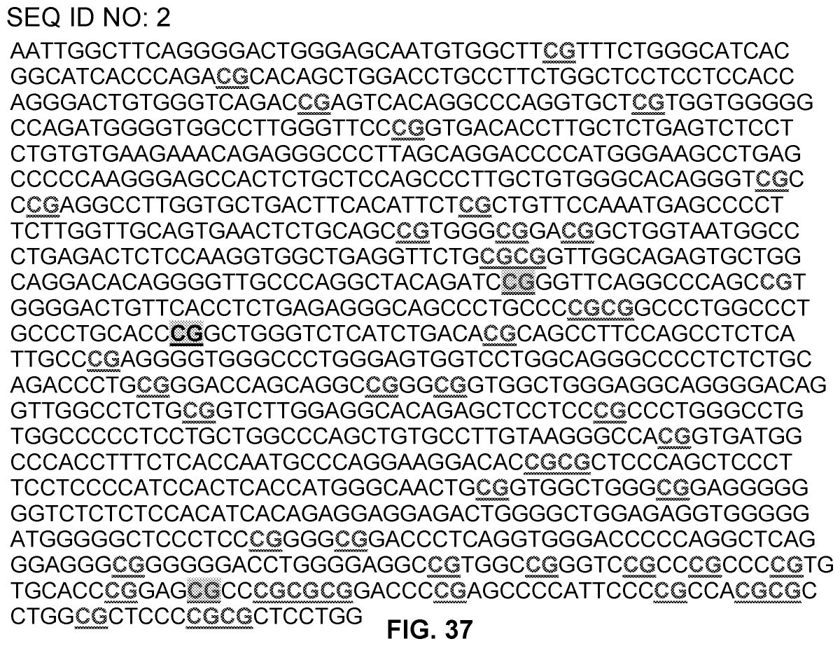

The CpG dinucleotide within the AHRR gene can be selected from the group of: cg05575921, cg21161138, cg26703534, zcg04987734; zcg20732076; zcg18917643; zcg25998745; zcg23779890 and any combination thereof. As can be seen in FIG. 37, the base pair sequence provides sufficient information to allow someone skilled in the art to create primers to identify methylation levels at any of the individual CpG sites for AHRR identified above.

The biological sample can be any biological fluid or tissue sample. In some embodiments, the biological sample can be blood. The blood can be peripheral blood. The biological sample can be a mononuclear cell pellet prepared from the biological sample. The biological sample can be a cell. The cell can be a monocyte, leukocyte, and/or red blood cell. The biological sample can be a tissue or portion thereof. The biological sample can be from diseased tissue, blood, organ and/or area. The biological sample can be from a non-diseased tissue, blood, organ, and/or area. Additionally in some embodiments, the biological sample can be drawn from an affected tissue, or a tissue more directly affected by the exposure, condition, or medication.

The method can further include an amplifying step after any or all of the contacting step(s). The method can further include a sequencing step performed after the amplifying step. With this in mind, the method can take the form of a PCR (polymerase chain reaction) assay and/or pyrosequencing (or other next generation sequencing) assay. It will be appreciated that although next generation sequencing techniques are specifically described in relation to the methods, that Sanger sequencing methods can also be used where sequencing is desired. These assays can be performed as real-time PCR or sequencing assays. By amplifying and/or sequencing the bisulfite treated DNA bound to the probe, the methylation status of CpG dinucleotide(s) can be determined.

The probes and/or primers described herein can be labeled using techniques know to those of skill in the art. The labels can be fluorescent dyes, radiolabels, enzymes, spectral colorimetric labels or plastic beads. Specific examples of these labels and labeling techniques will be known to one of skill in the art. The label can be coupled directly or indirectly to a component (e.g. primer or probe) of the detection methods provided herein. One of skill in the art will be able to determine the specific label to include, based on inter alia, the sensitivity required, ease of conjugation with the particular component, stability required, available instrumentation, etc. Methods of detecting and quantify such labels are generally known to those of skill in the art. Thus, for example, where the label is a radioactive label, means for detection can include a scintillation counter or photographic film as in autoradiography. Where the label is optically detectable (e.g. fluorescent labels), typical detectors include microscopes, cameras, phototubes, and photodiodes. Many other detection systems suitable for use with the methods described herein will be appreciated by one of ordinary skill in the art in view of the descriptions herein. In particular, indirect detection of methylation status may be based on methods of gene expression that can provide a “mirror image” index of the moderating effect of MTHFR on change in methylation in response to MMA exposure.

The methods described herein can further include the step of treating the subject in need thereof. An advantage of the assay described herein is the identification and selection of a patient population that prior to the development of this assay was unable to be treated because it was not known to exist. For example, this assay can allow identification of a nascent smoking population that before this assay went undetected for reasons described elsewhere herein. In some embodiments, the method can further include the step of administering behavioral therapy, psychiatric treatment, psychotherapy, pharmaceuticals, and combinations thereof. In some embodiments, the pharmaceutical can be or include bupropion, varenicline, nortriptyline, clonidine, Nicoderm® nicotine or similar products. In some embodiments, the subject is an unknown smoker or has been exposed to an MMA, such as tobacco or other compound in tobacco smoke as determined by an assay described herein.

In some embodiments, the method can further include the step of administering folate to the subject in need thereof. In some embodiments, the method can further include the step of administering one or more vitamins to the subject in need thereof. In some embodiments, the method described herein can be used in conjunction with treatment of a subject with folic acid supplementation alone, folic acid in combination with other vitamins and minerals; or, supplementation focused on providing of 5-methyltetrahydrofolate (5-MTHF), either alone or in combination with other vitamins and/or minerals.

### Kits

Also provided herein are kits for determining the methylation status of at least one CpG dinucleotide. The kits can include at least one first oligonucleotide probe, wherein the first oligonucleotide probe is complementary to a nucleotide sequence that comprises a CpG dinucleotide in the methylene tetrahydrofolate reductase (MTHFR) gene, wherein the first oligonucleotide probe detects either the unmethylated CpG dinucleotide or the methylated CpG dinucleotide. The kit can further include a second oligonucleotide probe, wherein the second oligonucleotide probe is complementary to a nucleotide sequence that comprises a CpG dinucleotide within the aryl hydrocarbon receptor repressor (AHRR) gene.

The CpG dinucleotide can be a CpG dinucleotide in the MTHFR gene. In some embodiments, the CpG dinucleotide in the MTHFR gene can be selected from the group of: cg01134491, cg01226883, cg02978542, cg05228408, cg05265975, cg08269394, cg08869383, cg10221637, cg11276438, cg12751404, cg14032528, cg14472778, cg17514528, cg17745097, cg18187189, cg21864959, cg22877851, cg23068701, cg23088157, cg23226134, cg23952195, cg25628740, cg27012203, and any combination thereof. The CpG dinucleotide in the MTHFR gene can be selected from the group consisting of: cg02978542, cg08269394, cg12751404, cg14032528, cg23068701, cg23226134, cg23952195, and any combination thereof. In some embodiments, the CpG dinucleotide in the MTHFR gene can be cg14032528.

The CpG dinucleotide within the AHRR gene can be selected from the group of: cg05575921, cg21161138, cg26703534, and any combination thereof.

The kit can also include any additional primers and/or probes necessary to conduct any PCR, sequencing, or other amplification or identification of a probe bound to a specific CpG dinucleotide. In some embodiments, primers can be labeled such that they can function as both CpG dinucleotide probe and primers. In other embodiments, primers can be used with a CpG dinucleotide specific probe (which can be optionally labeled).

The kit can include a solid substrate to which the first oligonucleotide probe, the second oligonucleotide probe, or the first oligonucleotide and the second oligonucleotide probe can be attached. The substrate is a polymer, glass, semiconductor, paper, metal, gel, or hydrogel. Suitable substrates will be appreciated by those of ordinary skill in the art. The substrate (and any attached probes) can be configured as a microarray or microfluidic chip.

The kit can include a detectable label. The labels can be fluorescent dyes, radiolabels, enzymes, spectral colormetric labels or plastic beads.

The kit can include an array. With this in mind, also provided herein are arrays, including microarrays, which can be used to detect one or more of the CpG dinucleotides and/or methylation thereof described elsewhere herein present in the DNA in a biological sample. In an array, one or more probes (e.g. oligonucleotide probes) can be attached to or operatively linked to a substrate in essentially discrete locations on the substrate. The discrete locations on the substrate where the probe(s) are attached to or operatively linked are individually referred to as a feature of the array and collectively as features. The features can be arranged in any desired arrangement on the substrate. The arrangement can be such that each feature has its own coordinate so as to allow identification of the probe and/or a CpG dinucleotide or methylation thereof detected at any given discrete location in the array according to the coordinate of the feature. These arrays can also be referred to as “ordered arrays”. The features can be arranged on the substrate to be 0.01 nm to 1 cm apart from another feature on the substrate. A single feature can contain a single probe (singleplex) or can contain more than one probes (multi-plex).

The substrate can be solid or semi-solid. The substrate can be rigid or be flexible. The substrate can contain one or more specialized layers that affect the functionality or performance of the array. The substrate can be two-dimensional or three-dimensional. The substrate can be made of glass, such as silicon dioxide or borosilicate; plastic, such as polystyrene, nylon, polyvinylidene difluoride; a fibrous material, such as cellulose, carboxy methyl cellulose, or nitrocellulose; a gel, such as agarose, a hydrogel, or polyacrylamide, The substrate can be formed into any desired shape, including but not limited to a square, a rectangle, a circle, a cube, a rectangular prism, or other regular or irregular polygonal shape or its corresponding three-dimensional shape. The substrate can have a length, a width, a height, a radius, and/or a diameter. The length of the substrate can range from about 1 μm to about 10 cm. The height of the substrate can range from about 1 μm to about 10 cm. The width of the substrate can range from about 1 μm to about 10 cm. The radius of the substrate can range from about 1 μm to about 10 cm. The diameter of the substrate can range from about 1 μm to about 10 cm.

The substrate can contain a single layer to which the probe is attached or operatively linked. In these embodiments, the substrate can also be referred to as the surface layer. In other embodiments, the substrate can contain more than one layer. In embodiments with more than one layer, the layer to which the probe is attached or operatively linked is referred to as the surface layer. The surface layer can be modified to affect the interaction and/or reduce non-specific binding between a probe and the substrate and/or the probe and the biomarker. In some embodiments, surface layer is modified to enhance the interaction between the probe and the surface layer and/or the interaction between the probe and its corresponding biomarker. The modification of the surface layer can also reduce non-specific binding by the probe and/or the CpG dinucleotide.

In some embodiments, the surface layer is modified with a chemical modification. Suitable chemical modifications include but are not limited to reactive hydroxide groups, reactive primary, secondary, tertiary, and/or quaternary amine groups, a monolayer of a reactive antibody including but not limited to anti-glutathione S-transferase (anti-GST) antibodies, reactive epoxide groups, reactive methacrylate groups, aldehyde reactive groups, reactive A/G proteins that bind immunoglobulins, and 3-D film coatings, which are polymeric coatings containing activated covalent binding sites. In some embodiments, 3-D film polymeric coatings include, but are not limited to, polysaccharides and hydrophilic polymers. In some embodiments, the 3-D film activated covalent binding sites include, but are not limited to, N-hydroxy succamide esters. The surface layer can be modified to be positively charged, neutral, or negatively charged. The surface layer can be modified to be hydrophilic, hydrophobic, or to contain a mix of hydrophobic and hydrophilic regions. In some embodiments, the modifications are patterned on the surface layer to form discrete functionalized areas to which the probe is attached or operatively-linked. In some embodiments having mixed hydrophobic and hydrophilic regions, the hydrophilic regions are separated by hydrophobic regions. In other embodiments, having mixed hydrophobic and hydrophilic regions, the hydrophobic regions are separated by hydrophilic regions.

In some embodiments, the surface layer is a gel, including but not limited to agarose, a hydrogel, or polyacrylamide. In some embodiments the substrate contains multiple discrete gel surface layers. These gel surface layers are also referred to as pads and can be arranged on the substrate in an ordered arrangement such that each gel pad is a feature of the array. In some embodiments, the same probe(s) are attached to or operatively linked to all the gel pads forming the surface layer of the substrate. In other embodiments, at least two of the gel pads have at least one different probe attached or operatively linked thereto.

The substrate can be configured to have one or more three dimensional discrete indentations or depressions in the surface layer. The probe(s) can be attached or operatively linked to the indentation. The three dimensional indentions can be square, rectangular, round, or irregular shaped. The three dimensional indentations can form wells or channels. One or more indentations can be connected to another indentation by a three dimensional connector channel extending between the one or more wells. In some embodiments, the connector channel is a microfluidic channel. In some embodiments, the microfluidic channel contains wicking paper. A dimension of the indentation can range from about 1 μm to about 10 cm. In some embodiments, a length of an indentation ranges from about 1 μm to about 10 cm. In further embodiments, a width of an indentation can range from about 1 μm to about 10 cm. In additional embodiments, a height of an indentation can range from about 1 μm to about 10 cm. In other embodiments, the radius of an indentation can range from about 1 μm to about 10 cm. In further embodiments, the diameter of an indentation can range from about 1 μm to about 10 cm. The indentations can be so dimensioned so as to hold a specific volume. In some embodiments, the specific volume ranges from about 1 nL to about 1,000 mL. In a single array, the indentations can all be about the same dimension. In other embodiments, at least two of the indentations differ in at least one dimension. Any surface of an indentation can be modified as described above with respect to modification of the surface layer.

The substrate can also contain additional layers beneath the surface layer and within the substrate. The additional layers can be directly beneath the surface layer or contain other layers, such as the substrate, between the additional layer and the surface layer. The additional layer can improve the signal to noise ratio, affect signal production produced by the binding of a probe to a CpG dinucleotide or other substrate, and affect other properties or performance parameters of the array. In some embodiments the additional layer is a dielectric layer. The dielectric layer can improve the reflection of the signal produced upon binding of a probe and a CpG dinucleotide.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Introduction

Smoking has enormous implications for population-level health in the US. Smoking consistently ranks as the single greatest preventable cause of morbidity and mortality in the U.S., with documented effects on increased risk for cancer, type 2 diabetes, chronic obstructive pulmonary disease, and obesity (Centers for Disease Control and Prevention, 2008). Smoking causes nearly half a million US deaths per year along with higher population rates of preventable, serious illness (Mokdad, Marks, Stroup, & Gerberding, 2004). This burden is not shared equally. Among US residents, people of African descent suffer worse outcomes for smoking-related illnesses (Control & Prevention, 2010; Haiman et al., 2006), even after adjusting for common covariates such as socioeconomic status [SES] and healthcare access. This suggests that examination of clinical samples of African American samples could create a better understanding through which other factors interact with smoking to alter morbidity and mortality.

The Use of cg05575921 as an Indicator of Smoking Intensity.

A significant barrier to a better understanding has been the lack of good biomarkers of cigarette consumption. However, over the past several years, a large number of groups have demonstrated that smoking is associated with changes in DNA methylation [Philibert et al., 2012, 2013; Zellinger, et al., 2013; Shenker, et al., 2013; Monick, et al., 2012; Joubert et al., 2012; Breitlig, et al., 2011]. These studies have shown that whereas longer-term smoking in older adults is associated with widespread and extensive remodeling of DNA methylation, particularly at loci associated with inflammation or toxin metabolism. In contrast, the extent of genome wide remodeling is much less extensive for nascent smokers or those with less smoking history (Philibert et al., 2012; 2013), with changes often confined to the aryl hydrocarbon receptor repressor (AHRR). This observation led to the proposal that measurement of a single CpG residue on AHRR (cg05575921) might suffice for accurate detection of nascent smoking in many cases, allowing an enhanced role for testing in pediatric care (Beach et al., 2015)

The biochemical basis for the sensitivity of the methylation status of cg05575921 can be due to the large amounts of dioxin and poly aromatic hydrocarbons (PAH) found in tobacco smoke (Talhout et al., 2011). When these and other toxins from cigarette smoke are inhaled and absorbed by blood passing through the lungs, there is a robust cellular response by white blood cells and pulmonary macrophages. In particular, this response includes activation of the aryl hydrocarbon receptor (AHR), a nuclear receptor which is the key regulator of the xenobiotic response, which in turn leads to the activation of CYP1A1, CYP1A2 and CYP1B1. Unfortunately, the unrestrained activity of these and other cytochromes by AHR can result in generation of reactive oxygen species (ROS), catabolism of key cellular components and potentially carcinogenesis (Ramadoss, Marcus, & Perdew, 2005). Therefore, a second nuclear receptor, AHRR, serves as a feedback modulator of the effects of AHR activation by competing for the obligate heterodimer partner of AHR, the aryl hydrocarbon nuclear translocator, and for DNA binding motifs for AHR. The enhancer region of AHRR that includes cg05575921 plays a key role in this increase in AHRR activity. Zeilinger and colleagues have shown the cg05575921 demethylation is associated with the disassociation of repressive protein complex and the recruitment of transcriptional activators. As a result, excessive activity of the xenobiotic pathway is avoided and as a direct consequence, methylation of AHRR, and specifically the region around cg05575921, is an exquisitely sensitive indicator of smoking intensity.

Do Individual Differences Influence AHRR Response to Smoking?

Our understanding of the methylomic response to smoking is still incomplete. In particular, one question left unresolved by prior research is whether folate-metabolism related individual differences may be consequential for degree of, or rapidity of, demethylation in response to smoking. Activated methyl groups are required for DNA methylation, regulation of chromatin structure, and maintenance of DNA methylation patterns when cells divide. Accordingly, individual differences affecting availability of activated methyl groups may, in turn, affect maintenance of DNA methylation patterns in replicating cells (Jones & Liang, 2009). Because smoking is associated with substantial reductions in plasma folate (Gabriel et al., 2006; Okumura & Tsukamoto, 2011) as well as in red blood cells (C. J. Piyathilake et al., 1992; Walmsley, Bates, Prentice, & Cole, 1999) and buccal cells (C. J. Piyathilake et al., 1992; C J Piyathilake et al., 1995), even after correcting for folate intake (C J Piyathilake et al., 1995), it would be expected that most smoking effects on methylation would be in the direction of demethylation. If so, individual differences that further reduce availability of activated methyl groups could potentially amplify the effect of smoking on genomewide demethylation (Jacob et al., 1998), potentially amplifying as well the initial response of nascent smokers. Identifying sources of such individual differences could enhance the ability of methylation based bio-markers to even more sensitively identify smokers as well as correctly quantify level of cigarette consumption based on methylation markers.

The Role of MTHFR in Methylation Maintenance.

Because methylene tetrahydrofolate reductase (MTHFR) is the rate-limiting enzyme in the methyl cycle, a pathway that is important to folate metabolism, it could be a source of potential individual differences. Smooth functioning of this cycle maintains adequate levels of S-adenosylmethionine (SAM) in cells, providing a readily available methyl group donor for numerous methylation related reactions (Stover, 2009), especially genomic methylation. Individual differences that could be associated with reduced MTHFR activity or expression could lead to reductions in DNA methylation repair over time or increase the rate of loss of methylation in response to stimuli associated with demethylation or hyper methylation such as cigarette smoking. Conversely, when MTHFR is expressed more or more active, methylation of homocysteine could be enhanced, potentially blunting the impact of external factors like smoking that would otherwise lead to demethylation (Frosst et al., 1995; Stover, 2009) or contributing to hyper methylation in response to perturbing agents.

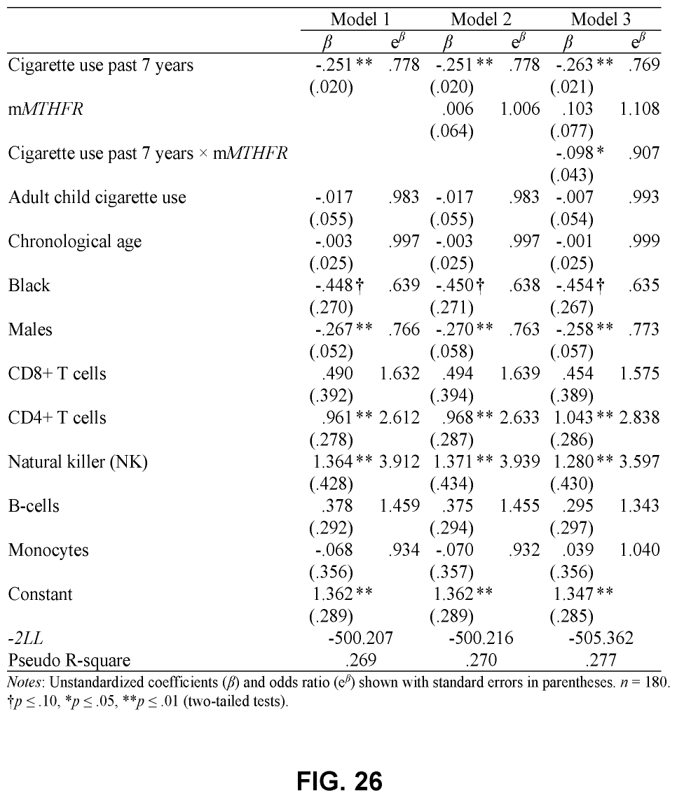

The MTHFR gene is located on chromosome 1, region 1p36.3, and contains 11 exons and three alternative transcription start sites, all in the vicinity of the first exon (Goyette et al., 1994) See FIG. 26.

Several polymorphisms that may potentially influence MTHFR activity have been identified, with one of the most commonly cited variant being rs1801133, a C to T substitution at position 677 (C677T) that converts a codon for alanine to one for valine (167A to 167V) (Sibani et al., 2000). The 167V isoform of MTHFR is less active, more thermolabile and is associated with hypohomocyteinemia (Goyette & Rozen, 2000). Not all ethnicities are affected equally; rs1801133 is in strong population disequilibrium with the frequency of T allele in African Americans (about 20%) being almost half of that in those of Northern European ancestry (Sherry et al., 2001).

MTHFR and Folate Metabolism in Disease.

Lower activity of MTHFR reduces the synthesis of 5-methylTHF and hence remethylation of homocysteine, an outcome associated with global hypomethylation (Castro et al., 2004; Yi et al., 2000). In tissue culture, low folic acid levels lead to increased formation of micronuclei, indicating increased rates of chromosome breakage. Further, the low activity MTHFR TT genotype leads to increased micronuclei formation under low folate conditions (Kimura, Umegaki, Higuchi, Thomas, & Fenech, 2004), and is associated with global DNA hypomethylation in circulating leukocytes, perhaps in interaction with level of folate (Friso et al., 2002; Stern, Mason, Selhub, & Choi, 2000). Variation in folate metabolism also has been linked to increased vulnerability for a number of disease states, including risk of cardiovascular disease, multiple cancers, and neural tube defects (Erickson, 2002; Holmquist, Larsson, Wolk, & de Faire, 2003; Lamprecht & Lipkin, 2003). In addition, the MTHFR TT genotype has been shown to increase risk for a wide variety of neuropsychiatric disorders including depression and schizophrenia (Gilbody, Lewis, & Lightfoot, 2007; Lewis, Zammit, Gunnell, & Smith, 2005) as well as a broad behavioral phenotype for psychopathology (Peerbooms et al., 2011). As described in this Example, there could be a role for MTHFR, folate metabolism, and their combination for use in better predicting DNA methylation in response to smoking, with potential implications for vulnerability to longer-term disease outcomes associated with smoking.

Evidence suggests that variation in methyl donor availability exists and that it could alter the epigenomic response to smoking, which makes it difficult to make meaningful predictions regarding cumulative exposure based on evaluation only on methylation of the cg05575921 loci of AHRR. The relationship between genetic and epigenetic differences in MTHFR and demethylation of cg05575921 in response to nascent smoking, among other things, are evaluated in this Example.

The Specific Hypotheses (H) tested were as follows:

H1: Early cumulative smoking will be associated with changes in cg05575921 methylation, even after controlling for the impact of sex, cell type variation, SES, methylation of MTHFR, and in the SHAPE sample genotype at MTHFRC677T.

H2: Because it should down-regulate gene expression, greater methylation of the first exon of MTHFR will moderate (e.g., intensify) the association of smoking with demethylation of cg05575921, even after controlling for the main effect of smoking and mMTHFR, and the effect of cell-type variability, sex, SES-risk, and in the SHAPE sample, genotypic variation at MTHFRC677T.

H3: Effects will replicate after removing potential false negatives, or those very recently initiating smoking, e.g., those denying smoking across all three earlier waves of self-report data collection, but who test positive for cotinine at the time of the blood draw.

H4: Patterns seen at cg05575921 will replicate at other loci on the AHRR gene previously found to be associated with smoking: cg21161138 and cg26703534.

Materials and Methods

Overview.

The data utilized are from two longitudinal research projects: the Strong African American Healthy Adult Project (SHAPE; n=322; see Brody et al., 2013 for a full description) and the Adults in the Making project (AIM; n=294; see Brody, Yu, Chen, Kogan, & Smith, 2012 for a full description). All procedures and protocols conducted in these studies were approved by the University of Georgia Institutional Review Board.

Initial Sample.

For the AIM sample, 496 youth were randomly recruited from public-school lists in six rural Georgia counties; none of which overlapped with those included in SHAPE. Again, the recruitment strategy was designed to obtain a representative sample of African American youth and families. The primary caregivers worked an average of 38.5 hr per week; 42.0% of the youth lived below federal poverty standards, with a median family income of $2,016 per month. Of the 376 youth with complete self-report data at ages 17-19, 293 (77.9%) provided blood samples at age 22. These 293 participants (107 men and 186 women) constituted our AIM sample. Comparisons of their families with the AIM families not included in the present study revealed no differences on any variables.

Replication Sample.

The SHAPE participants and their families were drawn from nine rural counties in Georgia, and so resided in small towns and communities in which poverty rates are among the highest in the nation and unemployment rates are above the national average (Proctor & Dalaker, 2003). Participants were selected randomly from lists of students that schools provided. Recruitment procedures were designed to obtain a sample of African American youth that was representative of the communities from which it was drawn; no other selection criteria were used. For the 399 (79.8%) who provided blood samples, primary caregivers worked an average of 39.9 hr per week, 42% of the participants lived below federal poverty standards, and they had a median family income of $1,732 per month. The sample for the present study was composed of 368 participants (167 men and 201 women) for whom blood was drawn at age 20 and for whom data was successfully genotyped for MTHFR polymorphism. Comparisons between these 368 participants and the 31 participants who were excluded because of incomplete data on genotyping revealed no significant differences on any of the key study variables or confounder variables. Fifty-six participants reported no cigarette use from ages 17-19, but subsequently were found to have cotinine levels >1.

For both samples, all data were collected in participants' homes using standardized protocols. Interviews were conducted privately, with no other family members present or able to overhear the conversation. Participants were compensated $100 at each wave of data collection. Informed-consent forms were completed at all data-collection points.

Self-reported smoking and potential confounder variables were assessed in SHAPE and AIM when the youth were approximately 17, 18, and 19 years of age (for AIM, average ages were Wave 1=17.05; Wave 2=18.48; Wave 3=19.12; for SHAPE, average ages were Wave 1=17.01; Wave 2=17.58; Wave 3=18.44). Subsequently, participants in each cohort provided antecubital blood samples (drawn at age 20 in SHAPE and at age 21 in AIM), and these were used to assess methylation genome-wide as well as to check cotinine for secondary analyses excluding potential false negatives and recent initiators.

Measures

SES-Risk.

Caregiver reports collected at the same time as youth reports of smoking were used to create our measure of concurrent Socio-economic risk. SES risk was assessed across six indicators. Each indicator was scored dichotomously (0 if absent, 1 if present). Cumulative SES risk was defined as the average number of risk factors across the three assessments, yielding an index with a theoretical range of 0 to 6 (M=2.33, SD=1.35). The six risk indicators were (a) family poverty, defined as being below the poverty level, taking into account both family income and number of family members; (b) primary caregiver non-completion of high school or an equivalent; (c) primary caregiver unemployment; (d) single-parent family structure; (e) family receipt of Temporary Assistance for Needy Families; and (f) income rated by the primary caregiver as not adequate to meet all needs.

Cigarette Consumption.

At each wave of data collection in both SHAPE and AIM participants were asked “In the past month, how much did you smoke cigarettes?” Response options included: 0—None at all; 1—Less than 1 cigarette a day; 2-1 to 5 cigarettes a day; 3—About a half a pack a day; 4—About a pack a day; 5—About 1 and a half packs a day; 6—About 2 packs a day.

Methylation.

Certified phlebotomists drew antecubital blood samples of whole blood (30 ml) from each participant and shipped it to a lab in Iowa the same day for preparation. At the lab the blood tubes were inspected to ensure anticoagulation and aliquots of blood were diluted, mononuclear cell pellets were separated from the diluted blood specimen by density-gradient centrifugation, and the mononuclear cell layer was removed from the tube using a transfer pipette, resuspended, and frozen at −80 degree C. until use. Genomic DNA was prepared using a QiaAmp (Qiagen, Germany) according to manufacturer's directions. Atypical DNA yield for each mononuclear cell pellet was between 10 and 15 μg.

The Illumina (San Diego, Calif.) HumanMethylation450 Beadchip was used to assess genome-wide DNA methylation. Participants were randomly assigned to 12 sample “slides/chips” with groups of 8 slides being bisulfite converted in a single batch, resulting in five “batches/plates.” A replicated sample of DNA was included in each plate to aid in assessment of batch variation and to ensure correct handling of specimens. The replicated sample was examined for average correlation of beta values between plates, resulting in average correlations greater than 0.99. Prior to normalization, methylation data were filtered based on these criteria: 1) samples containing 1% of CpG sites with detection p-value >0.05 were removed, 2) sites were removed if a beadcount of <3 was present in 5% of samples and 3) sites with a detection p-value of >0.05 in 1% of samples were removed. More than 99.76% of the 485,577 probes yielded statistically reliable data. Probes associated with known SNPs or close to SNPs are annotated within Illumina allowing the presence of SNPs in regions of interest to be examined.

Quantile Normalization of Methylation Data.

Quantile normalization methods with separate normalization of Type I and Type II assays in the Illumina array produce marked improvement in detection of relationships by correcting distributional problems inherent in the manufacturers default method for calculating R (i.e., p=M/(M+U+100; where M and U are methylated and unmethylated signal intensities, respect (Pidsley et al., 2013). Accordingly, in the current investigation all loci across all plates were quantile normalized concurrently, separating methylated and unmethylated intensities, and using the wateRmelon (2013) R package (Pidsley and others, 2013) to institute the “dasen” function. This method equalizes the backgrounds of Type I and Type II probes prior to normalization and includes between-array normalization of Type I and Type II probes separately.

Identifying and Correcting for Chip and Batch Effects.

As demonstrated by Sun and associates (Sun et al., 2011), quantile normalization typically reduces, but may not eliminate, batch and chip effects. Accordingly, after cleaning and quantile normalizing the data, all samples were examined for batch and chip effects. The distribution of quantile normalized average R values for all samples in each chip and batch were contrasted with all others using a box and density plot to indicate both the mean and confidence intervals around the mean in each case. The results of this examination indicated that both batch and chip effects were eliminated through quantile normalization. Absence of plate effects was confirmed via direct examination of the sample replicated across plates.

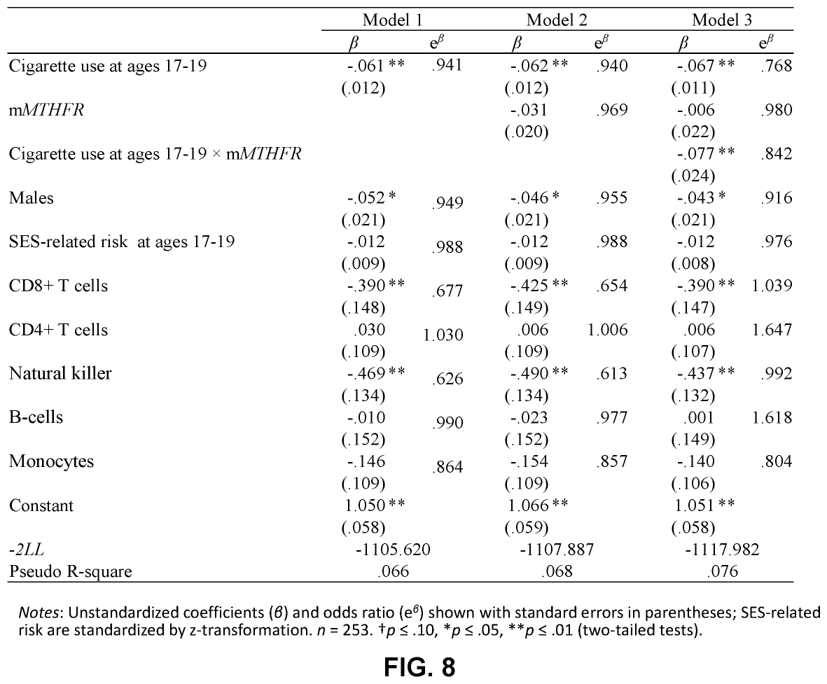

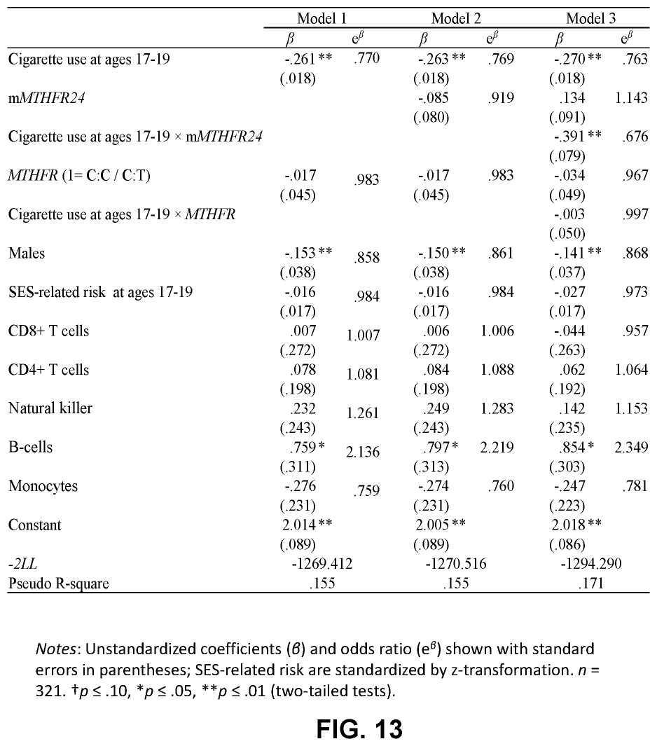

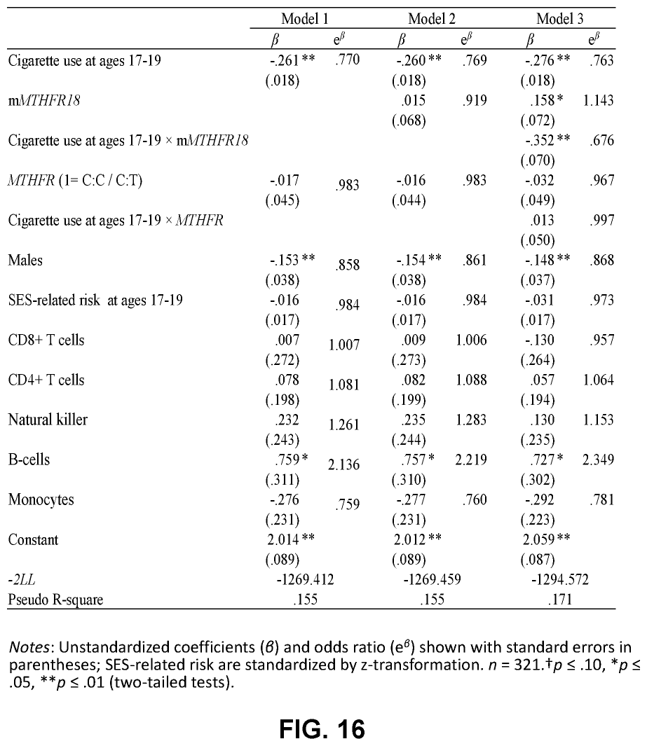

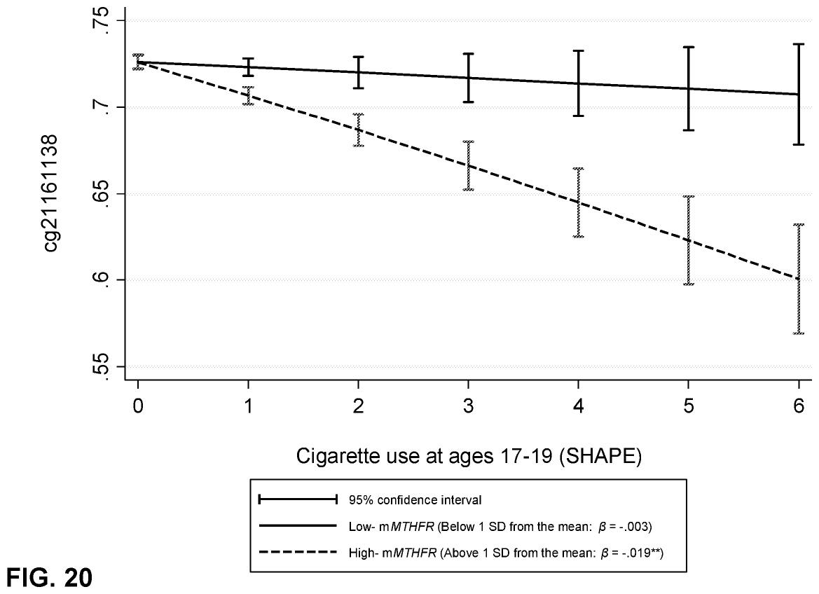

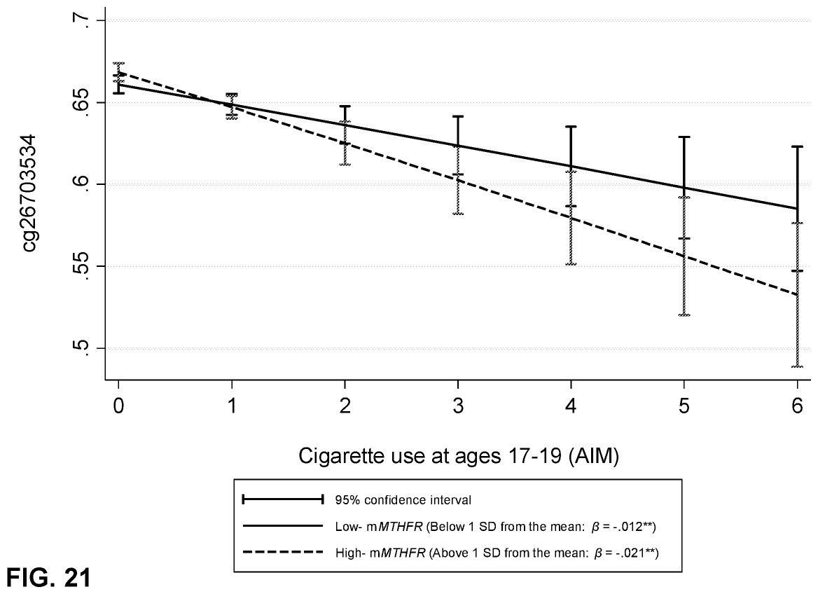

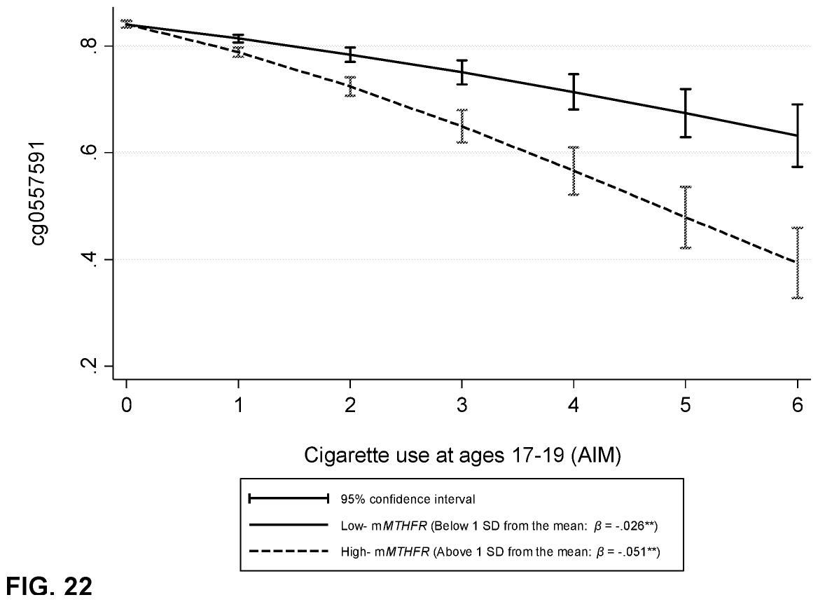

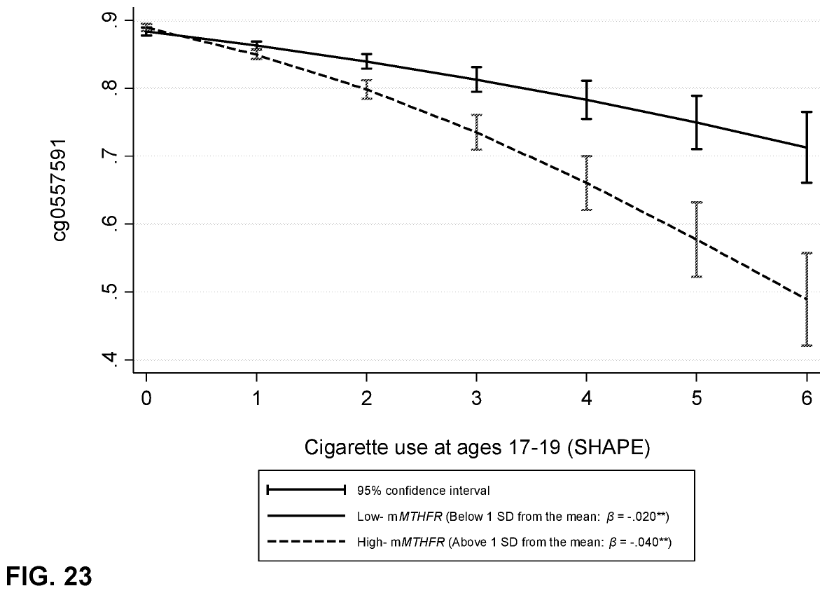

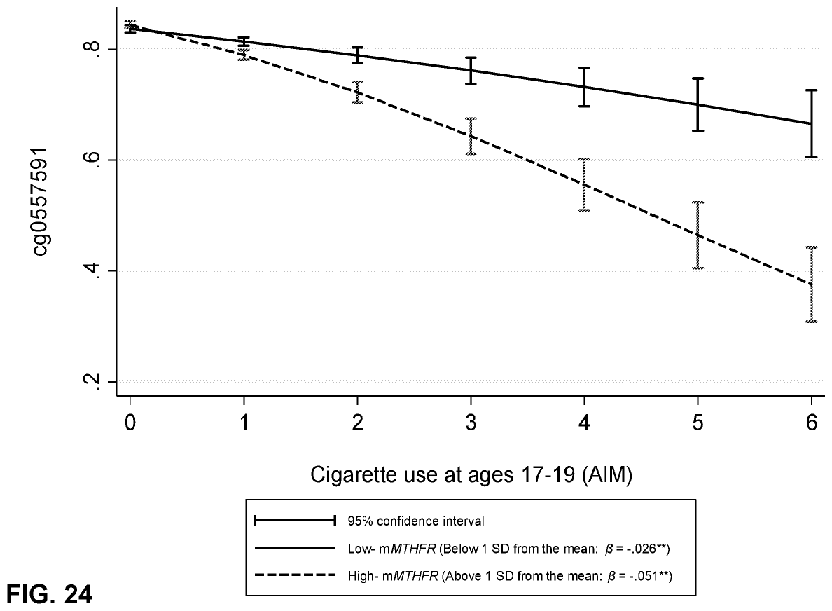

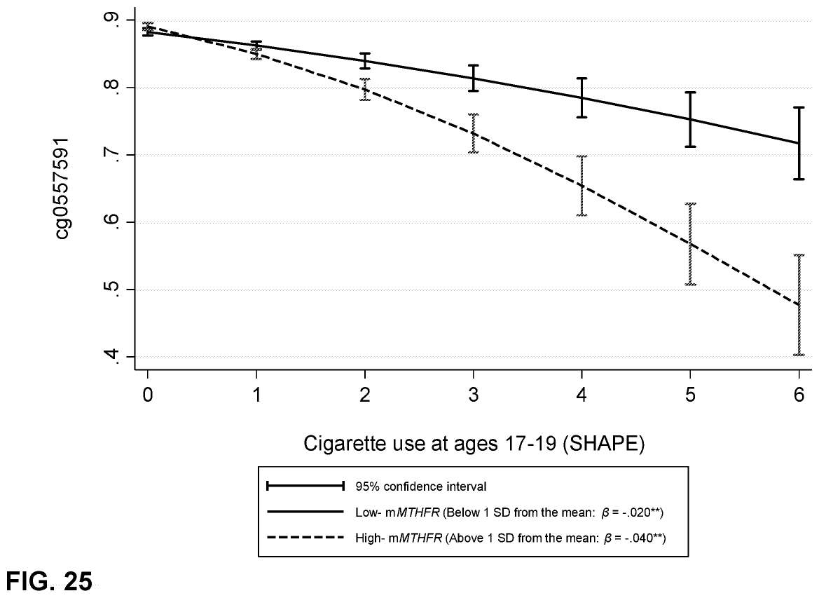

Index of mMTHFR. The understanding of the epigenetic regulation of gene expression is still in its infancy. However, Brenet and colleagues demonstrated that methylation of the first exon is involved, in which hypomethylation of the first exon can be associated with a relatively large effect on gene expression (LOR=−2.8) and experimentally manipulated demethylation at the first exon also producing a large effect (Brenet et al., 2011). To create the index of methylation of MTHFR (mMTHFR), all loci annotated as being associated with the first exon of MTHFR were utilized. The resulting set of seven loci (cg02978542, cg08269394, cg12751404, cg14032528, cg23068701, cg23226134, cg23952195) were all also annotated as being on the CpG island for MTHFR and as being promoter associated. The composite index of MTHFR methylation was calculated by averaging the standardized scores of the seven retained CpGs following quantile normalization. That is, mMTHFR=mean(zcg02978542, zcg08269394, zcg12751404, zcg14032528, zcg23068701, zcg23226134, zcg23952195). So, all loci contributed equally to the index of mMTHFR. In FIGS. 8-25 the results are presented that use a mean index of 7 loci (denoted as mMTHFR) or all 24 loci annotated as being associated with MTHFR (denoted as mMTHFR24) or as the mean of 18 loci with no potential for local cis effects due to SNP variation (denoted as mMMTHFR18).

Assessing and Controlling Proportion of Cell Types in Mixed Cell Populations.

Mononuclear cell pellets of the sort used in the current investigation are comprised of several different cell types (e.g., primarily T-helper and cytotoxic cells, monocytes, B cells, and natural killer (NK) cells, (Reinius, et al., 2012). Accordingly, individual differences in cell types were controlled for using an approach developed by Houseman and colleagues (2012) through the “EstimateCellCounts” function in the minfi Bioconductor package.

Genotyping.

Youths' participating in SHAPE were genotyped for MTHFR^{C677T}, a mutation associated with decreased activity of MTHFR (Frosst et al., 1995). DNA was obtained at age 16 using Oragene™ DNA kits (Genetek; Calgary, Alberta, Canada). Youths rinsed their mouths with tap water, then deposited 4 ml of saliva in the Oragene sample vial. The vial was sealed, inverted, and shipped via courier to a central laboratory in Iowa City, where samples were prepared according to the manufacturer's specifications. The prepared DNA was robotically dispensed into 384 optimal PCR trays. Subsequently, they were genotyped for the MTHFRC677T (rs1801133) using standard Taqman® reagents (Applied Biosystems (ABI), Foster City, Calif.) and an existing ABI 7900 HT Genotyping system. Of the 374 participants contributing saliva, successful genotyping for MTHFRC677T was achieved for 368 participants.

Plan of analysis. Beta regression analyses was used in all cases because the dependent variable (degree of methylation) is calculated as a ratio of methylated to unmethylated loci ranging from zero to one, and so typically fails the assumption of having a normal distribution (Dolzhenko & Smith, 2014). Beta regressions, utilizing the beta distribution, were introduced by Ferrari & Cribari-Neto (Ferrari & Cribari-Neto, 2004), and are useful when the dependent variable is bounded, or when it has a ceiling or floor effect, introducing a non-normal distribution for the dependent variable.

On step 1 of each beta regression, the main effect of cumulative self-reported smoking across three years in early adulthood was entered. The impact of self-reported smoking in a multivariate context was examined, controlling for the effect of sex, cell-type variation, and in the case of the SHAPE sample, genotype at MTHFR^{C677T}. On the second step the additional main effect of mMTHFR was examined, again accounting for multivariate influences. On the third step the effect of adding the interaction term, mMTHFR×cumulative smoking was added, and in the case of the SHAPE sample, the interaction term of genotype at MTHFR^{C677T} with cumulative smoking to control any interaction of genotype and smoking was also added. This basic series of steps was repeated across both the SHAPE and AIM samples as well as for the reduced samples resulting from the removal of potential false negatives, e.g., anyone reporting no cigarette use, but nonetheless, found to have elevated cotinine at the time of the blood draw. Provided in FIGS. 8-25 are similar regression analyses for the two additional loci on AHRR (cg21161138 and cg26703534) that typically show main effect associations with nascent smoking.

Results.

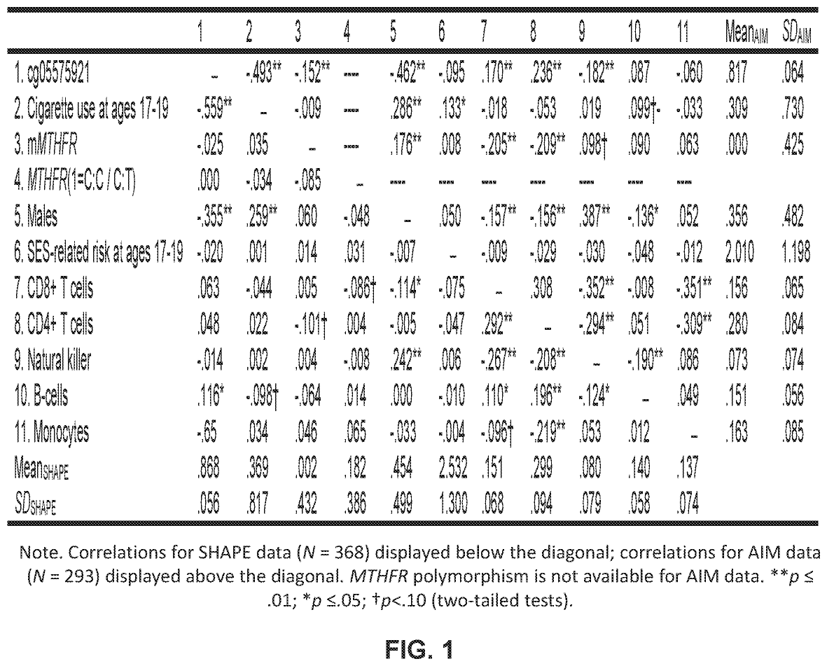

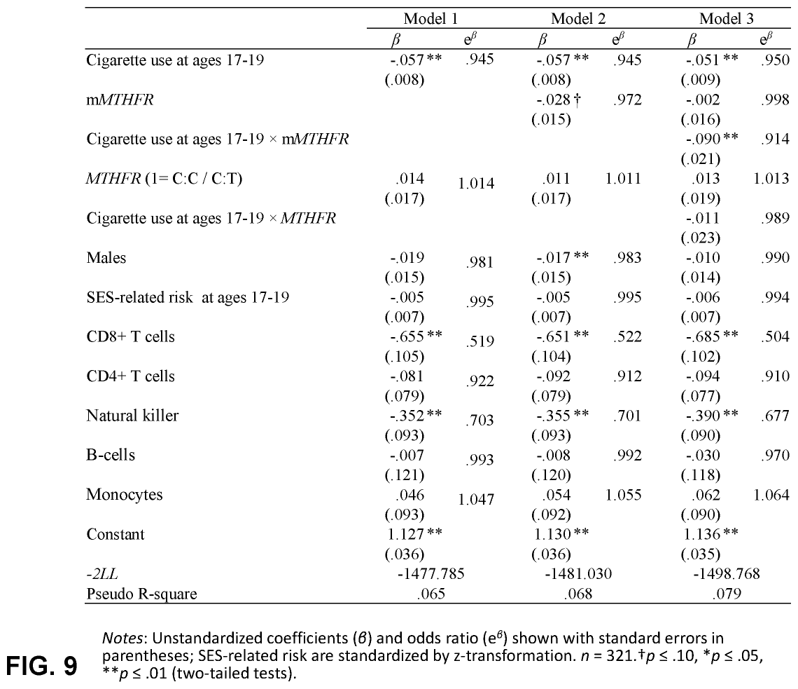

Results are demonstrated in FIGS. 1-25. Hardy-Weinberg. Genotype at MTHFR^{C677T} was determined for each youth as previously described. Of the sample, 1.1% were homozygous for the low activity T allele (TT), 17.1% were heterozygous (CT), and 81.8% were homozygous for the high activity C allele (CC). The distribution of alleles did not deviate from Hardy-Weinberg equilibrium (p=0.731, ns). In FIGS. 8-9, the demographic characteristics and main study variables were compared for carriers of the T allele vs. CC homozygotes for the SHAPE sample and found no significant demographic differences as a function of genotype.

H1: Early Cumulative Smoking Will be Associated with Changes in cg05575921 Methylation.

FIG. 1 presents means, sds, and intercorrelations for all demographic and major study variables. As can be seen in FIG. 1, cumulative smoking across three years in late adolescence was correlated with cg05575921 in both samples. In the SHAPE sample, the MTHFR^{C677T} genotype was not associated with mMTHFR and was found to be infrequent (18.2% had any T allele, 1.1% (4 cases only) were homozygotes). Significant correlations were found between self-reported smoking and cg05575921 and sex, with marginal correlations with cell-type, and a non-significant association with SES-risk in SHAPE and a significant association in AIM.

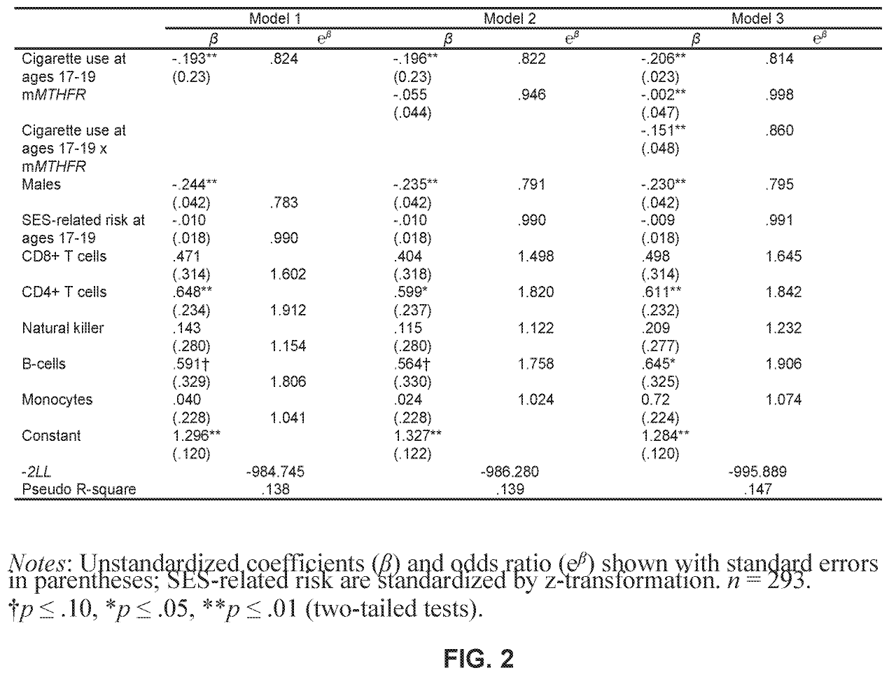

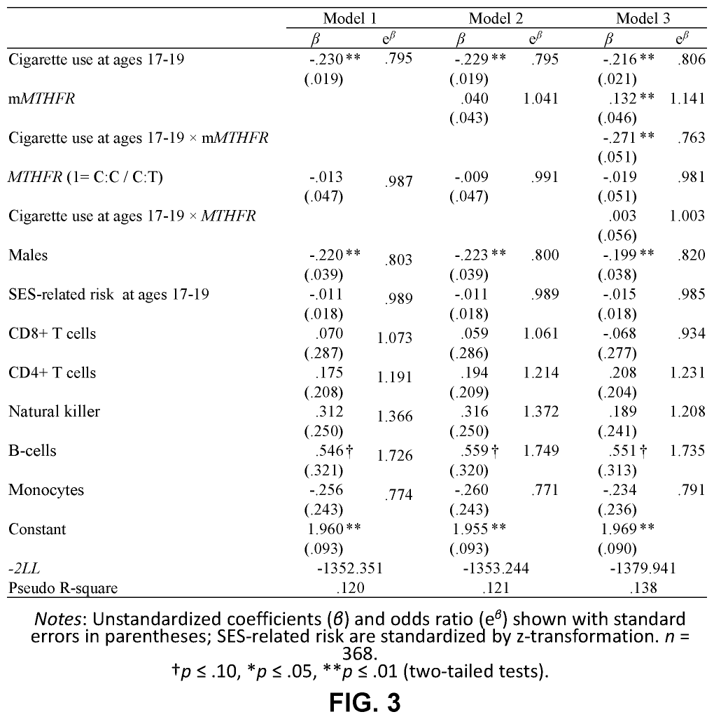

As can be seen in FIGS. 2 and 3, column 1, even when covariates for sex, SES risk, and cell-type variation were included, cumulative smoking across three years in late adolescence continued to be strongly correlated with demethylation of cg05575921, indicating that the association of smoking with demethylation of cg05575921 is robust to covariates, including variation in cell-types comprising the PBMC pellets for each participant.

H2: Greater mMTHFR Will Moderate (i.e., Intensify) the Impact of Smoking on Demethylation of cg05575921.

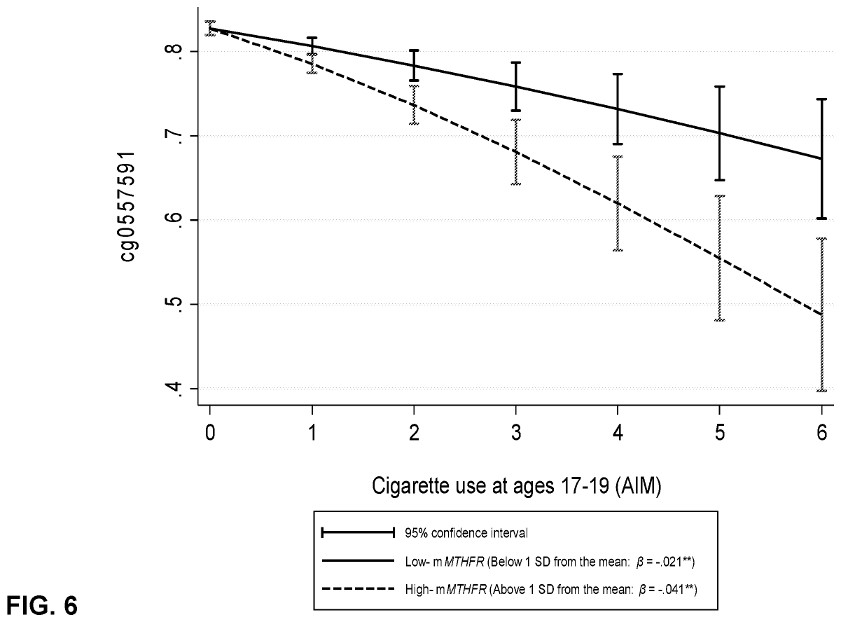

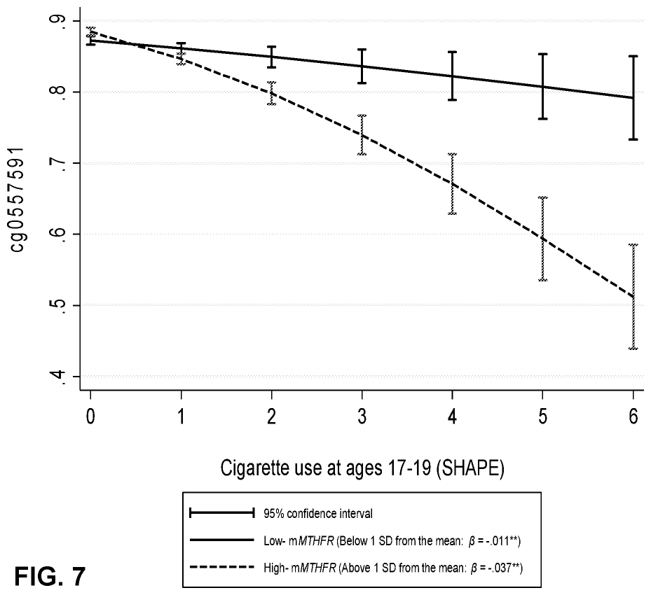

In column 2 of FIG. 2, the main effect of mMTHFR was entered and exerted no significant main effect on methylation at cg05575921. In step 3, the interaction of mMTHFR and level of smoking was entered into the model, which resulted in a significant interaction effect in each sample. The significant interaction effect is plotted in FIG. 6 (for the AIM sample) and FIG. 7 ((for the SHAPE sample), showing that, as anticipated, greater methylation at MTHFR was associated with a stronger effect of smoking on demethylation of cg05575921 in response to cumulative smoking. As can be seen in FIG. 3, in the SHAPE sample there was no significant interaction effect of genetic variation at MTHFR667 with smoking showing that variation at this locus did not account for the observed moderating effect of mMTHFR on the impact of smoking.

H3: Effects Will Replicate in the Reduced Sample that Excludes Those Denying Smoking Despite Testing Positive for Cotinine.

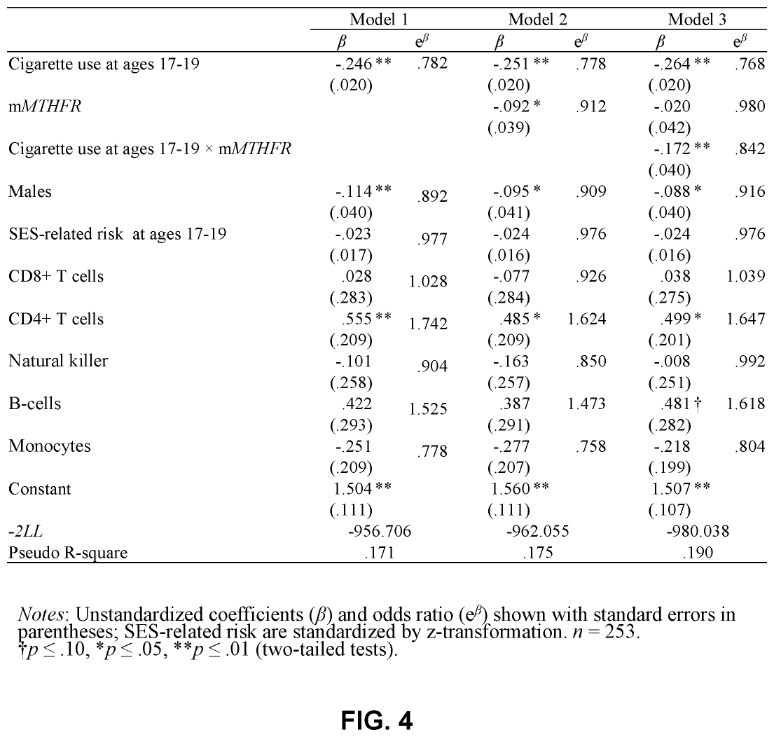

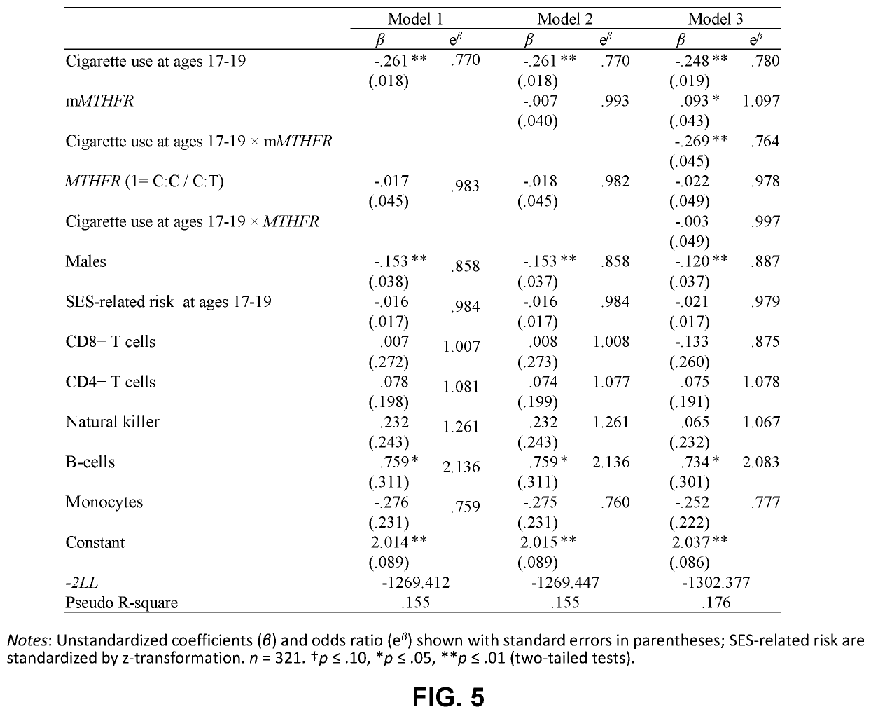

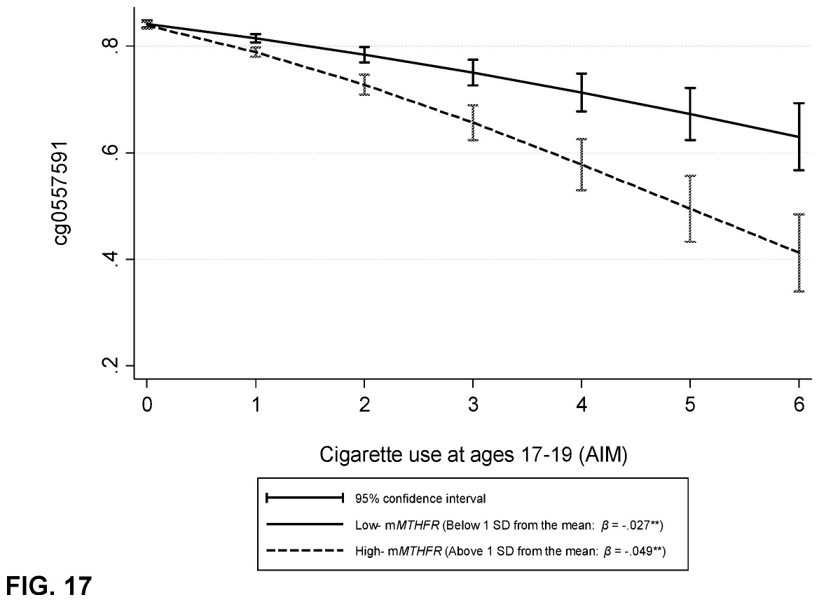

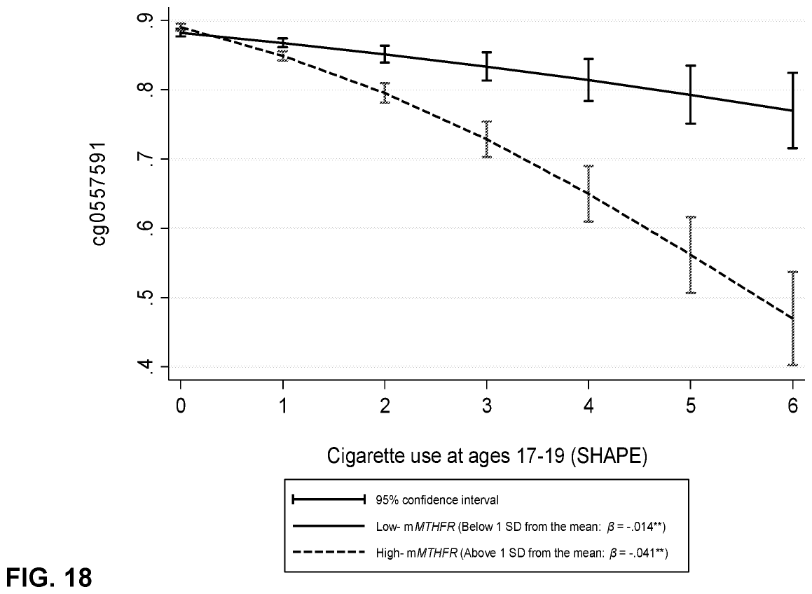

To examine the effect of eliminating potential false negatives, we eliminated cotinine positive participants who denied smoking at all prior waves of data collection, thereby reducing error due to underreporting or recent onset of smoking. Accordingly, we replicated the findings reported above using only the cotinine verified participants in each sample (FIG. 4 for AIM and FIG. 5 for SHAPE). In each case the results replicated exactly. There is a robust effect of smoking on methylation at cg05575921 controlling potential confounding variables, and the interaction of mMTHFR with smoking continues to be significant in the direction of an intensified demethylation response among those with greater methylation of MTHFR. Graphical explication of the interaction effects for FIG. 4 and FIG. 5, replicating the pattern previously portrayed in FIGS. 6 and 7 are shown in FIGS. 17 and 18).

H4: Effects Will Replicate for Other AHRR Loci Typically Found to be Associated with Smoking: cg21161138 and cg26703534

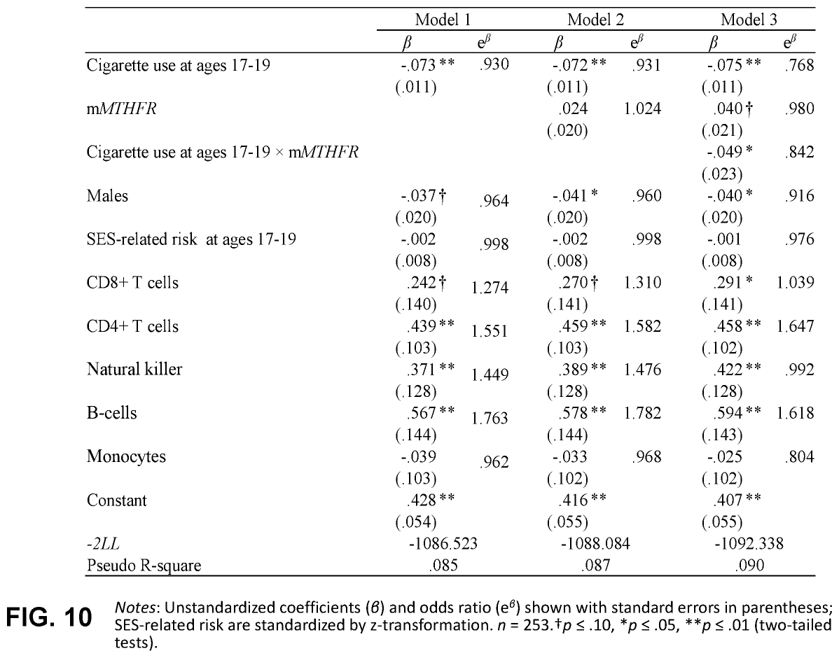

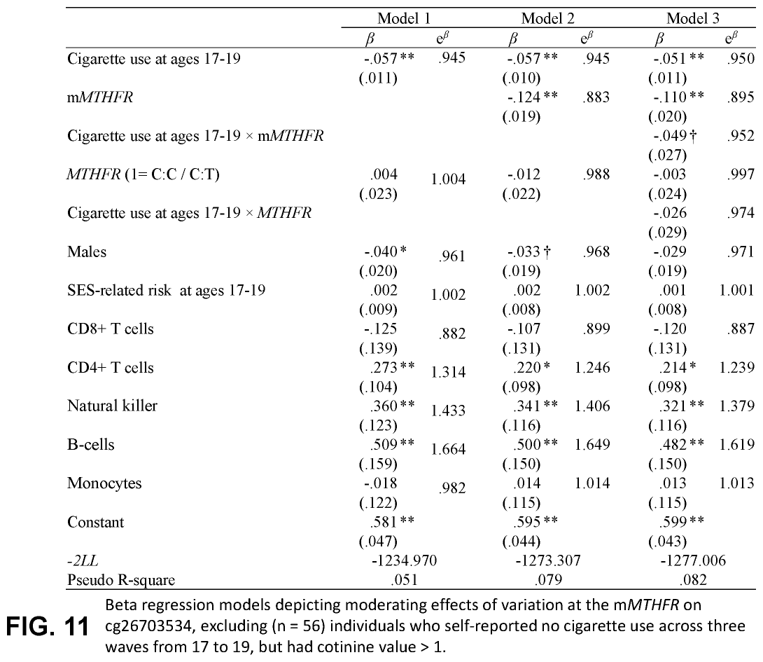

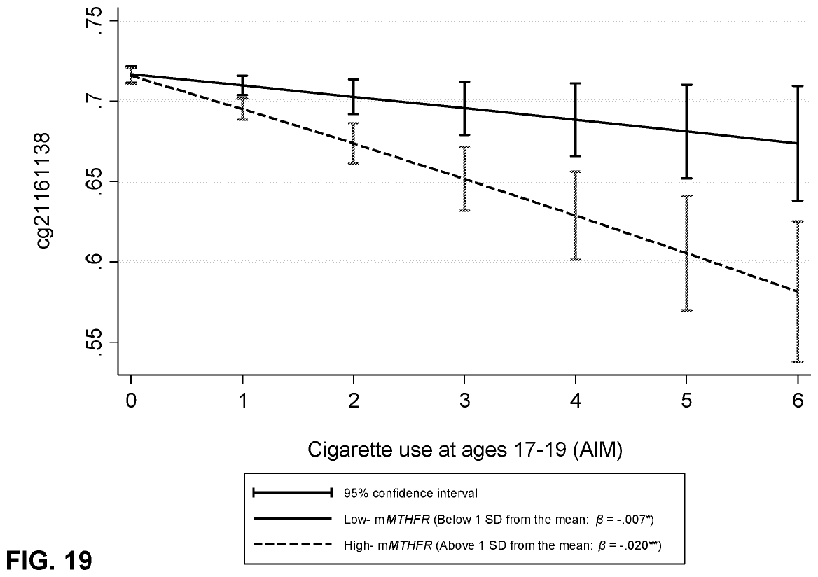

To examine replication of a similar interaction pattern for the two other loci on AHRR previously found to be associated with nascent smoking (R. Philibert, Beach, Li, & Brody, 2013), we re-ran regression analyses substituting methylation at these loci as outcomes in place of cg05575921. In both samples there was a significant main effect of self-reported smoking on demethylation at the additional AHRR loci. Significant moderation of smoking's impact attributable to mMTHFR was found for both additional AHRR loci in the AIM sample (See FIGS. 8 and 10) and for one of the additional loci (cg21161138) in the SHAPE sample (See FIG. 9). At the second locus (cg26703534), the interaction was marginal (p=0.067) in the SHAPE sample, albeit in the same direction (see FIG. 11). In all cases, the significant interaction of mMTHFR with smoking resulted in an intensification of the demethylation response among those with greater methylation of MTHFR. The graphical explication of the significant interaction effects are shown in FIGS. 19, 20, and 21).

Discussion

The initiation of smoking initiates a strong response by the xenobiotic pathway, leading to demethylation, particularly at cg05575921, but also secondarily at other CpG sites. This effect has been replicated in a number of samples (Elliott et al., 2014; Monick et al., 2012; R. Philibert et al., 2013; R. A. Philibert, Beach, & Brody, 2012; Shenker et al., 2012; Zeilinger et al., 2013; Zhang et al., 2014) and appears to be robust across ethnicities (Dogan et al., 2015). This impact of smoking on cg05575921 methylation is not surprising given the key biological role played by AHRR in response to the types of toxins commonly found in cigarette smoke. This Example adds to this finding by showing for the first time that the impact of smoking on demethylation of cg05575921 is potentially qualified by an epigenetic regulatory motif: methylation of the first exon of MTHFR.

Despite the success of cg05575921 as an early and sensitive indicator of incipient smoking and changes in smoking status, it is not perfectly correlated with self-reported smoking, a limitation likely due, in part, to problems inherent in self-report (e.g. Philibert and others, 2013). When self-report errors are minimized, for example, by excluding individuals whose cotinine level suggests they are smokers even though they deny smoking, the correlation of smoking with cg05575921 methylation typically increases. However, there is sufficient variability in methylation levels among youth reporting similar, low levels of smoking, to suggest the presence of other individual difference factors moderating the impact of nascent smoking experience on demethylation of cg05575921. Such sources of variability will be particularly important when accurate quantification of prior smoking exposure is a primary goal rather than simply differentiating smokers and non-smokers. As outlined above, a potential source of such variability is individual differences in the expression of MTHFR, a source regulated by methylation of MTHFR.

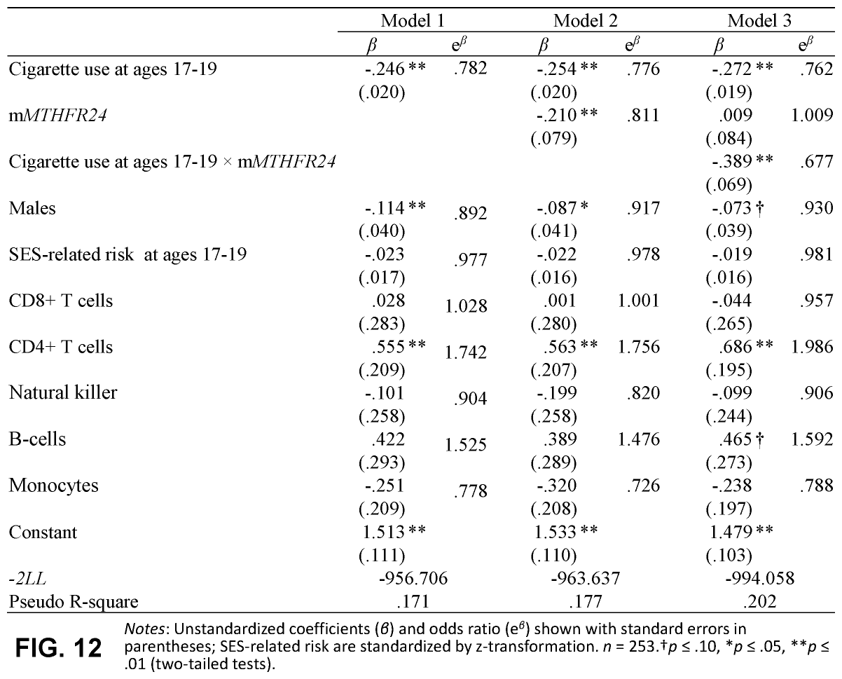

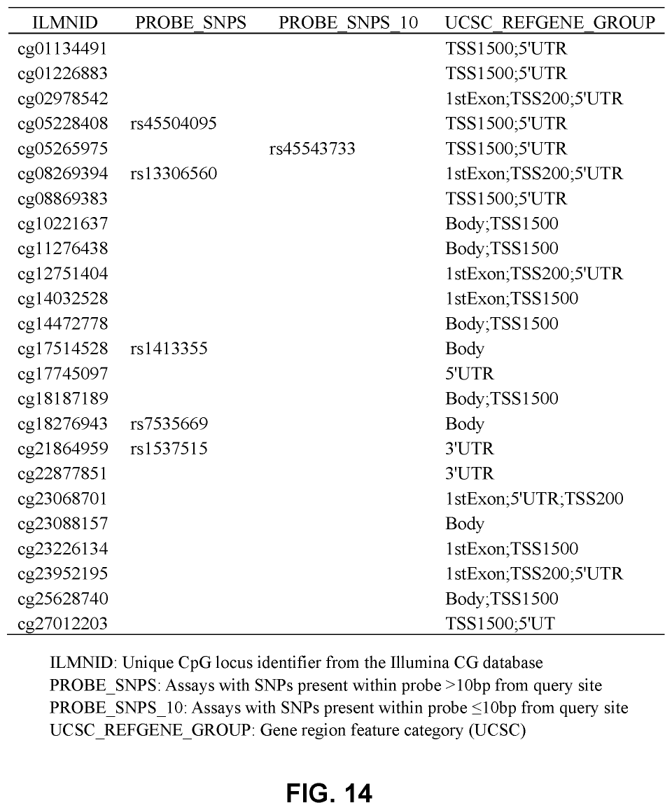

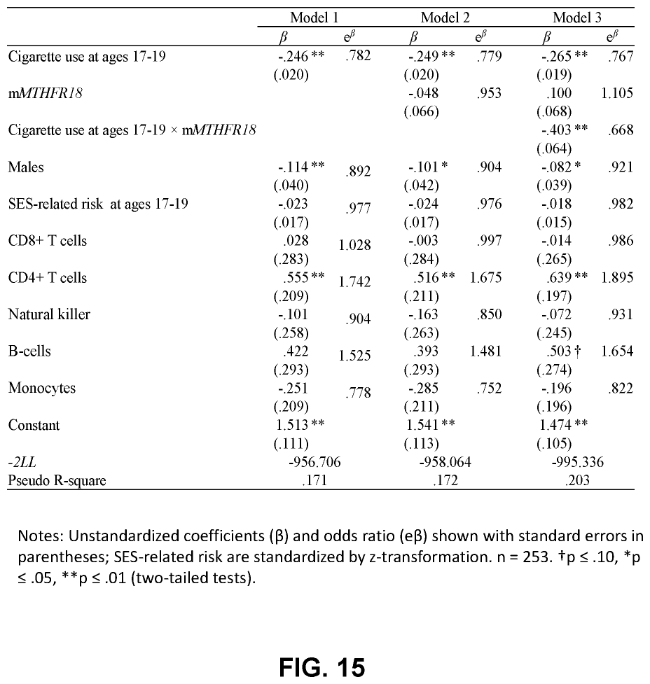

In the current investigation it was found in replicated evidence that for African American youth, the impact of self-reported smoking on demethylation of cg05575921 was intensified when methylation of the first exon of MTHFR was elevated. This is significant because it suggests that in the context of a strong stimulus favoring demethylation, like cigarette smoking, it is possible to observe direct regulatory impact of MTHFR methylation patterns on the epigenetic impact of smoking at AHRR. The interaction was predicted on the basis of expectations that methylation of a first exon would have a strong impact on reduced expression and hence reduced activity of a gene that is central to folate metabolism. Accordingly, elevated methylation of the first exon of MTHFR was expected to result in better maintenance of methylation at cg05575921 and so less apparent effect of smoking. It is noteworthy that the same pattern was replicated for two other AHRR loci known to be influenced by smoking. In addition, the effect using average methylation across all loci annotated as being associated with MTHFR regardless of location on the gene was examined and it was found that the pattern was again replicated, suggesting that observation of the pattern does not depend on restricting attention to the first exon (See FIGS. 12, 13, 22, and 23). Finally, when six loci that were on or close to known SNPs on MTHFR (see FIG. 14) were dropped from the index, overall MTHFR methylation continued to moderate the impact of smoking on methylation at cg05575921 (see FIGS. 15, 16, 24, and 25.), suggesting that the moderating effect was not due to cis effects of cryptic genotypic variation within MTHFR.

Given the centrality of the folate cycle to a broad range of biological activities, it is possible that the index of mMTHFR used in the current study is itself correlated with broader patterns of methylation, and so with variability in other gene regulatory motifs across the genome, resulting in coordinated pattern of change. If so, it may be useful in future research, using larger samples, to examine such broader networks. By necessity this will include both broader sampling of methylation of other folate cycle genes, sampling of sera to determine folate levels, consideration of dietary factors including alcohol intake, and possibly gut biome interactions. Since many of these potentially independent influences will be, in fact, correlated, there should be an emphasis on studying cohorts for whom data on multiple levels of biological data are available.

It also will be informative to replicate the current results in older samples with longer histories of smoking to better understand the boundaries of the observed interaction effects. Likewise, given the widely varying frequency of genetic motifs influencing MTHFR activity, it will be useful to examine whether the same patterns are seen in samples with a greater proportion of carriers of the MTHFR^{C677T} polymorphism or other polymorphisms influencing MTHFR gene activity, e.g. Caucasian or Asian populations. Finally, it would be useful theoretically to tease apart the relative impact of recent vs cumulative smoking on demethylation of cg05575921 to determine whether both components are equally affected by MTHFR expression. A related question is whether methylation of MTHFR may moderate the response of cg05575921 to smoking cessation. Fortunately, because the loci utilized in the current investigation are included in the Illumina 450K array, the hypotheses advanced in the current study can be examined in any data set that includes both an assessment of smoking and Illumina 450K array data.

With regard to practical implications for assessment, the current results suggest that when demethylation of cg05575921 is used as a basis to quantify recent smoking history, it may be useful to examine mMTHFR as well. In the current investigation, the interaction term accounted for between 2.1 and 1.5% of the variance in demethylation of cg05575921 in response to smoking for the SHAPE and AIM cotinine verified samples respectively. Likewise, for all levels of consumption beyond zero and “less than one per day,” i.e., the two lowest levels of self-reported cigarette consumption, the level of demethylation in response to smoking differed significantly for those with higher vs. lower mMTHFR. For example, in the cotinine verified SHAPE sub-sample, someone with low mMTHFR who was found to have 80% methylation of cg0557592, would be estimate to been a heavy smoker, over the past several years, with cigarette consumption in the range of “1 and a half packs a day”; which is a 5 on our self-report scale. Conversely, someone with high mMTHFR with the same level of methylation at cg0557592 would be estimated to be a light smoker, with cigarette consumption in the range of “1 to 5 cigarettes a day”; a two on our self-report scale. Accordingly, there are potentially large differences in estimated cigarette exposure secondary to this regulatory motif, and the current results suggest the potential for improved prediction of demethylation in response to smoking exposure.

If the current findings are replicated more broadly, including replication in samples of longer-term smokers, it will be of interest to see if smoking's broader effects on demethylation genomewide are also moderated by regulatory motifs influencing the folate cycle. If so, such variability will be an attractive target for explication of individual differences in morbidity and mortality that are associated with smoking exposure. Specifically, it may be that cg05575921 demethylation can serve as a “canary in the coal mine,” providing an early forecast of patterns of epigenetic change likely to emerge with continued smoking, helping to identify individuals at greatest risk for smoking associated health complications.

### REFERENCES FOR EXAMPLE 1

Beach, S. R. H., Gerrard. M., Gibbons F. X., Brody, G. H., Philibert, R. A. (2015). A Role for Epigenetics in Broadening the Scope of Pediatric Care in the Prevention of Adolescent Smoking. Epigenetic Diagnosis & Therapy, 1(2), 91-97.
Brenet, F., Moh, M., Funk, P., Feierstein, E., Viale, A. J., Socci, N. D., & Scandura, J. M. (2011). DNA Methylation of the First Exon Is Tightly Linked to Transcriptional Silencing. PloS One, 6(1), e14524.
Castro, R., Rivera, I., Ravasco, P., Camilo, M. E., Jakobs, C., Blom, H. J., & De Almeida, I. T. (2004). 5, 10-methylenetetrahydrofolate reductase (MTHFR) 677C→T and 1298A→C mutations are associated with DNA hypomethylation. Journal of Medical Genetics, 41, 454-458.
Centers for Disease Control and Prevention. (2008). Smoking-attributable mortality, years of potential life lost, and productivity losses—United States, 2000-2004. MMWR: Morbidity and Mortality Weekly Report, 57(45), 1226-1228.
Control, C. f. D., & Prevention. (2010). Racial disparities in smoking-attributable mortality and years of potential life lost—Missouri, 2003-2007. MMWR. Morbidity and mortality weekly report, 59(46), 1518.
Dogan, M. V., Xiang, J., Beach, S. R., Cutrona, C., Gibbons, F. X., Simons, R. L., . . . Philibert, R. A. (2015). Ethnicity and Smoking-Associated DNA Methylation Changes at HIV Co-Receptor GPR15. Frontiers in psychiatry, 6.
Dolzhenko, E., & Smith, A. D. (2014). Using beta-binomial regression for high-precision differential methylation analysis in multifactor whole-genome bisulfite sequencing experiments. BMC Bioinformatics, 15(1), 1-8. doi:10.1186/1471-2105-15-215
Elliott, H., Tillin, T., McArdle, W., Ho, K., Duggirala, A., Frayling, T., Relton, C. (2014). Differences in smoking associated DNA methylation patterns in South Asians and Europeans. Clinical Epigenetics, 6(1), 4.
Erickson, J. D. (2002). Folic acid and prevention of spina bifida and anencephaly. 10 years after the US Public Health Service recommendation. MMWR. Recommendations and reports: Morbidity and mortality weekly report. Recommendations and reports/Centers for Disease Control, 51, 1-3.
Ferrari, S. L., & Cribari-Neto, F. (2004). Beta Regression for Modelling Rates and Proportions. Journal of Applied Statistics, 31(7), 799-815. doi: 10.1080/0266476042000214501
Friso, S., Choi, S. W., Girelli, D., Mason, J. B., Dolnikowski, G. G., Bagley, P. J., . . . Selhub, J. (2002). A common mutation in the 5, 10-methylenetetrahydrofolate reductase gene affects genomic DNA methylation through an interaction with folate status. Proceedings of the National Academy of Sciences, 99, 5606-5611.
Frosst, P., Blom, H. J., Milos, R., Goyette, P., Sheppard, C. A., Matthews, R. G., Van den Heuvel, L. (1995). A candidate genetic risk factor for vascular disease: a common mutation in methylenetetrahydrofolate reductase.
Gabriel, H. E., Crott, J. W., Ghandour, H., Dallal, G. E., Choi, S.-W., Keyes, M. K., Mason, J. B. (2006). Chronic cigarette smoking is associated with diminished folate status, altered folate form distribution, and increased genetic damage in the buccal mucosa of healthy adults. The American Journal of Clinical Nutrition, 83(4), 835-841.
Gilbody, S., Lewis, S., & Lightfoot, T. (2007). Methylenetetrahydrofolate Reductase (MTHFR) Genetic Polymorphisms and Psychiatric Disorders: A HuGE Review. Am. J. Epidemiol., 165(1), 1-13. doi:10.1093/aje/kwj347
Goyette, P., & Rozen, R. (2000). The thermolabile variant 677C-T can further reduce activity when expressed in CIS with severe mutations for human methylenetetrahydrofolate reductase. Human Mutation, 16(2), 132-138. doi:10.1002/1098-1004(200008)16:2<132::AID-HUMU5>3.0.CO;2-T
Goyette, P., Sumner, J. S., Milos, R., Duncan, A. M. V., Rosenblatt, D. S., Matthews, R. G., & Rozen, R. (1994). Human methylenetetrahydrofolate reductase: isolation of cDNA, mapping and mutation identification. Nature Genetics, 7(2), 195-200.
Haiman, C. A., Stram, D. O., Wilkens, L. R., Pike, M. C., Kolonel, L. N., Henderson, B. E., & Le Marchand, L. (2006). Ethnic and Racial Differences in the Smoking-Related Risk of Lung Cancer. New England Journal of Medicine, 354(4), 333-342. doi:doi:10.1056/NEJMoa033250
Holmquist, C., Larsson, S., Wolk, A., & de Faire, U. (2003). Multivitamin supplements are inversely associated with risk of myocardial infarction in men and women-Stockholm Heart Epidemiology Program (SHEEP). The Journal of Nutrition, 133, 2650-2654.
Jacob, R. A., Gretz, D. M., Taylor, P. C., James, S. J., Pogribny, I. P., Miller, B. J., Swendseid, M. E. (1998). Moderate Folate Depletion Increases Plasma Homocysteine and Decreases Lymphocyte DNA Methylation in Postmenopausal Women. The Journal of Nutrition, 128(7), 1204-1212.
Jones, P. A., & Liang, G. (2009). Rethinking how DNA methylation patterns are maintained. Nat Rev Genet, 10(11), 805-811.
Kimura, M., Umegaki, K., Higuchi, M., Thomas, P., & Fenech, M. (2004). Methylenetetrahydrofolate reductase C677T polymorphism, folic acid and riboflavin are important determinants of genome stability in cultured human lymphocytes. The Journal of Nutrition, 134, 48-56.
Lamprecht, S. A., & Lipkin, M. (2003). Chemoprevention of colon cancer by calcium, vitamin D and folate: molecular mechanisms. Nature Reviews Cancer, 3, 601-614.
Lewis, S. J., Zammit, S., Gunnell, D., & Smith, G. D. (2005). A meta-analysis of the MTHFR C677T polymorphism and schizophrenia risk. American Journal of Medical Genetics Part B: Neuropsychiatric Genetics, 135, 2-4.
Mokdad, A. H., Marks, J. S., Stroup, D. F., & Gerberding, J. L. (2004). Actual causes of death in the United States, 2000. JAMA, 291(10), 1238-1245. doi:10.1001/jama.291.10.1238
Monick, M. M., Beach, S. R., Plume, J., Sears, R., Gerrard, M., Brody, G. H., & Philibert, R. A. (2012). Coordinated changes in AHRR methylation in lymphoblasts and pulmonary macrophages from smokers. Am. J. Med Genet., 159B(2), 141-151. doi:10.1002/ajmg.b.32021
Okumura, K., & Tsukamoto, H. (2011). Folate in smokers. Clinica Chimica Acta, 412, 521-526.
Peerbooms, O. L., van Os, J., Drukker, M., Kenis, G., Hoogveld, L., De Hert, M., Rutten, B. P. (2011). Meta-analysis of MTHFR gene variants in schizophrenia, bipolar disorder and unipolar depressive disorder: evidence for a common genetic vulnerability? Brain, behavior, and immunity, 25, 1530-1543.
Philibert, R., Beach, S. R., Li, K.-M., & Brody, G. (2013). Changes in DNA methylation at the aryl hydrocarbon receptor repressor may be a new biomarker for smoking. Clinical Epigenetics, 5, 19-26. doi:10.1186/1868-7083-5-19
Philibert, R. A., Beach, S. R., & Brody, G. H. (2012). Demethylation of the aryl hydrocarbon receptor repressor as a biomarker for nascent smokers. Epigenetics, 7(11), 1331-1338.
Pidsley, R., Y Wong, C. C., Volta, M., Lunnon, K., Mill, J., & Schalkwyk, L. C. (2013). A data-driven approach to preprocessing Illumina 450K methylation array data. BMC Genomics, 14(1), 1-10. doi:10.1186/1471-2164-14-293
Piyathilake, C. J., Hine, R. J., Dasanayake, A. P., Richards, E. W., Freeberg, L. E., Vaughn, W. H., & Krumdieck, C. L. (1992). Effect of smoking on folate levels in buccal mucosal cells. International Journal of Cancer, 52(4), 566-569. doi:10.1002/ijc.2910520412
Piyathilake, C. J., Macaluso, M., Hine, R. J., Vinter, D. W., Richards, E. W., & Krumdieck, C. L. (1995). Cigarette smoking, intracellular vitamin deficiency, and occurrence of micronuclei in epithelial cells of the buccal mucosa. Cancer Epidemiology Biomarkers & Prevention, 4(7), 751-758.
Proctor, B. D., & Dalaker, J. (2003). Poverty in the United States: 2002. Washington D.C.: US Government printing office.
Ramadoss, P., Marcus, C., & Perdew, G. H. (2005). Role of the aryl hydrocarbon receptor in drug metabolism. Expert Opinion on Drug Metabolism & Toxicology, 1(1), 9-21. doi:10.1517/17425255.1.1.9
Shenker, N. S., Polidoro, S., van Veldhoven, K., Sacerdote, C., Ricceri, F., Birrell, M. A., . . . Flanagan, J. M. (2012). Epigenome-wide association study in the European Prospective Investigation into Cancer and Nutrition (EPIC-Turin) identifies novel genetic loci associated with smoking. Human Molecular Genetics. doi:10.1093/hmg/dds488
Sherry, S. T., Ward, M.-H., Kholodov, M., Baker, J., Phan, L., Smigielski, E. M., & Sirotkin, K. (2001). dbSNP: the NCBI database of genetic variation. Nucleic Acids Research, 29(1), 308-311. doi:10.1093/nar/29.1.308
Sibani, S., Christensen, B., O'Ferrall, E., Saadi, I., Hiou-Tim, F., Rosenblatt, D. S., & Rozen, R. (2000). Characterization of six novel mutations in the methylenetetrahydrofolate reductase (MTHFR) gene in patients with homocystinuria. Human Mutation, 15(3), 280-287. doi:10.1002/(SICI)1098-1004(200003)15:3<280::AID-HUMU9>3.0.CO;2-I
Stern, L. L., Mason, J. B., Selhub, J., & Choi, S. W. (2000). Genomic DNA hypomethylation, a characteristic of most cancers, is present in peripheral leukocytes of individuals who are homozygous for the C677T polymorphism in the methylenetetrahydrofolate reductase gene. Cancer Epidemiology Biomarkers & Prevention, 9, 849-853.
Stover, P. J. (2009). One-Carbon Metabolism-Genome Interactions in Folate-Associated Pathologies. The Journal of Nutrition, 139(12), 2402-2405. doi:10.3945/jn.109.113670
Talhout, R., Schulz, T., Florek, E., Van Benthem, J., Wester, P., & Opperhuizen, A. (2011). Hazardous compounds in tobacco smoke. International Journal of Environmental Research and Public Health, 8(2), 613-628.
Walmsley, C., Bates, C., Prentice, A., & Cole, T. (1999). Relationship between cigarette smoking and nutrient intakes and blood status indices of older people living in the UK: further analysis of data from the National Diet and Nutrition Survey of people aged 65 years and over, 1994/95. Public Health Nutrition, 2(02), 199-208. doi:doi: 10.1017/S1368980099000257
Yi, P., Melnyk, S., Pogribna, M., Pogribny, I. P., Hine, R. J., & James, S. J. (2000). Increase in plasma homocysteine associated with parallel increases in plasma S-adenosylhomocysteine and lymphocyte DNA hypomethylation. Journal of Biological Chemistry, 275, 29318-29323.
Zeilinger, S., Kihnel, B., Klopp, N., Baurecht, H., Kleinschmidt, A., Gieger, C., Illig, T. (2013). Tobacco smoking leads to extensive genome-wide changes in DNA methylation. PloS One, 8(5), e63812. doi:10.1371/journal.pone.0063812
Zhang, Y., Yang, R., Burwinkel, B., Breitling, L. P., Holleczek, B., Schöttker, B., & Brenner, H. (2014). F2RL3 methylation in blood DNA is a strong predictor of mortality. International Journal of Epidemiology. doi:10.1093/ije/dyu006

### Example 2

Example 1 is focused on nascent smokers, with relatively short smoking histories and limited cumulative smoke exposure. This begs the question of whether similar moderating effects would be seen for middle-aged adults with longer smoking histories. One might wonder, for example, whether the moderating effect is limited to younger samples, and whether mMTHFR is influenced by cumulative smoking exposure, reducing its independent predictive utility among older and more established smokers. Accordingly, to examine generalizability of mMTHFR effects on moderation of smoking related demethylation at CG05575921 among longer-term smokers, we examined the moderating effect of mMTHFR in a sample of middle-aged African Americans with longer smoking histories.

Details on prior waves of data collection for the FACHs project and general procedures utilized are described in prior publications (Simons and others, in press). Self-reported smoking status at prior waves were used to establish smoking history. At three waves of data collection across ages 41 to 48, middle-aged participants were asked at each wave of data collection “On average, how many cigarettes do you usually smoke per day?” Response options included: 0—none at all; 1—one to five cigarettes; 2—six to ten cigarettes; 3—eleven to fifteen cigarettes; 4-above sixteen cigarettes. Responses were averaged across waves to provide an index of overall smoking exposure across the seven-year period.

Blood Based Measures.

A certified phlebotomist drew five tubes of blood at each participant's home. All middle-aged participants who provided blood at wave 5 (67 males [mean age 48.60 yrs, SD=7.77] and 113 females [mean age 48.58 yrs, SD=9.15]), were eligible for inclusion in the sample.

Methylation Characterization.

Methylation was characterized as for Beach and others [2017]. Briefly, methylation was assessed using the Illumina (San Diego, Calif., USA) HumanMethylation450 Beadchip with standard tests for slide and plate effects and filtering for detection p-value >0.05 or a beadcount of <3. Prior to analyses, data were quantile normalized using the wateRmelon R package, dasen method [Pidsley and others 2013]. We created our index of mMTHFR using all loci annotated as being associated with the first exon of MTHFR.

Methylation Response at CG05575921.

Methylation Response at CG05575921 was characterized using droplet digital PCR (ddPCR). 1 μg of DNA from each subject was bisulfite converted using an EpiTect Fast 96 DNA Bisulfite kit (Qiagen, Hilden, Germany) according to the manufacturer's direction. The methylation ratio at CG05575921 in each bisulfite-treated sample was then determined using the Smoke Signature™ Assay (IBI Scientific, Peosta, Iowa) and a QX200 Droplet Digital PCR System™ (Bio-Rad, Hercules, Calif.) according to the manufacturer's protocols. This approach has been shown to provide excellent quantification of single locus methylation effects (Andersen et al. 2017).

Proportion of Cell Types.

We controlled for individual variation in the proportion of different cells types in the blood sample using standard methods. In particular, we used data on genomewide methylation to estimate individual differences in cell types using an approach developed [Houseman and others 2012] through the “EstimateCellCounts” function in the minfi Bioconductor package.

Analytic Plan.

In all cases, we used Beta regressions, as we have done previously [Beach and others, 2017]. Because the dependent variable (degree of methylation in CG05575921) is calculated as a ratio of methylated to unmethylated loci ranging from zero to one, it typically fails the assumption of having a normal distribution [Dolzhenko and Smith 2014]. Beta regressions, utilizing the beta distribution, were introduced by [Ferrari and Cribari-Neto 2004], to handle dependent variables that are bounded in this manner, accommodating the non-normal distribution that is introduced as a consequence.

Results of Extension to Middle-Aged Adults.

To examine generalizability of MTHFR's regulatory effect to an older sample we ran a series of beta regressions examining the regulatory impact of mMTHFR on response to smoking among the (N=180) primarily African American caregivers and partners who had been sampled and characterized for genomewide methylation as described above.

As can be seen in FIG. 26 which show the table of regression results, in model one we replicated the robust, inverse effect of smoking on CG05575921 (Philibert and others, 2013), even after controlling for our index of second smoke exposure via adult child smoking and controls for cell-type, age, and sex. Two indices of cell-type variation (CD4+ and NK) were significantly associated with CG05575921. Replicating and extending the previous finding by Beach et al [2017], in model 3, it can be seen that methylation of the first exon of MTHFR (mMTHFR) moderated smoking's effect on methylation in this middle-aged sample.

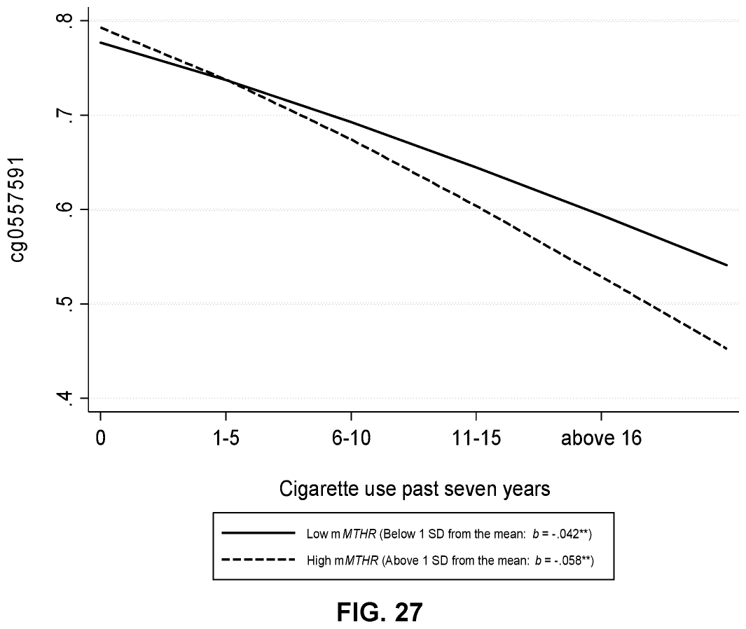

As can be seen in FIG. 27 (FIG. 1), the moderating effect of mMTHFR reflects a steeper slope relating smoking to cg05575921, e.g., more demethylation in response to smoking, among those with greater mMTHFR, whereas less mMTHFR was associated with a blunted effect of smoking on demethylation. This is the hypothesized pattern and replicates the findings reported for young adults by Beach and others [2017]. Accordingly, the previously described regulatory effect of mMTHFR on response to smoking was replicated for this middle-aged sample, with a significant effect of self-reported smoking on methylation of CG05575921, and a significant interaction effect of mMTHFR and smoking on CG05575921 in the predicted direction.

### Example 3

Examples 1 and 2 focus on the relatively limited set of loci whose methylation is significantly and profoundly affected among nascent smokers, e.g., loci that are reporters of acute exposure. However, continued exposure to cigarette smoke is known to exert increasingly widespread effects on patterns of genomewide methylation (Dogan et al., 2014), potentially leading to a range of disease states as broader and broader networks of gene expression are disrupted. Although the exact mechanism is not known, it is likely that the result of continuous exposure to the many toxins known to be contained in cigarette smoke exert this effect over time. Cigarette smoke contains both carcinogenic and non-carcinogenic toxins. This allows the use of the response to long-term cigarette smoking to test the hypothesis that mMTHFR will moderate the broader effect of multi-faceted long-term toxic exposures, amplifying alteration of methylation for loci that show a significant pattern of either increased methylation in response to longer-term exposure or that show a significant patter of decreased methylation in response to longer-term exposure.

To test this hypothesis previous findings by Dogan et al. (2014) were re-examined. Previously, it was shown that 910 loci were changed in response to long-term smoking, and this was true at a genomewide level of significance (Dogan et al., 2014). The list of affected loci is available online. It was reasoned that if MTHFR is regulating methylation response to toxic exposures, with longer-term exposure to cigarette smoke, individuals should show a broad pattern of effect that goes beyond the regulation of methylation for loci on AHRR demonstrated in Examples 1 and 2. It was hypothesized that there would be evidence of mMTHFR regulation of the broader DNA methylation response to the range of toxins present in cigarette smoke in the form of interactions of mMTHFR with smoking status, and this would be evident for the larger group of loci found to be hyper or hypo methylated at longer exposures. It was further hypothesized that mMTHFR would have a different effect at loci showing hypo methylation than at loci showing hyoer methylation, resulting in interaction effects with opposite signs. Using the same population as was used in the original finding, we reanalyzed the data in the same manner, except incorporating mMTHFR as a moderator (i.e. introducing an interaction term that included mMTHFR in the analyses).

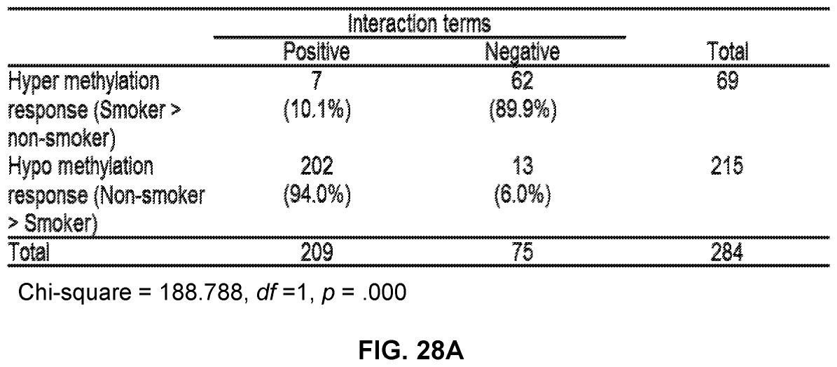

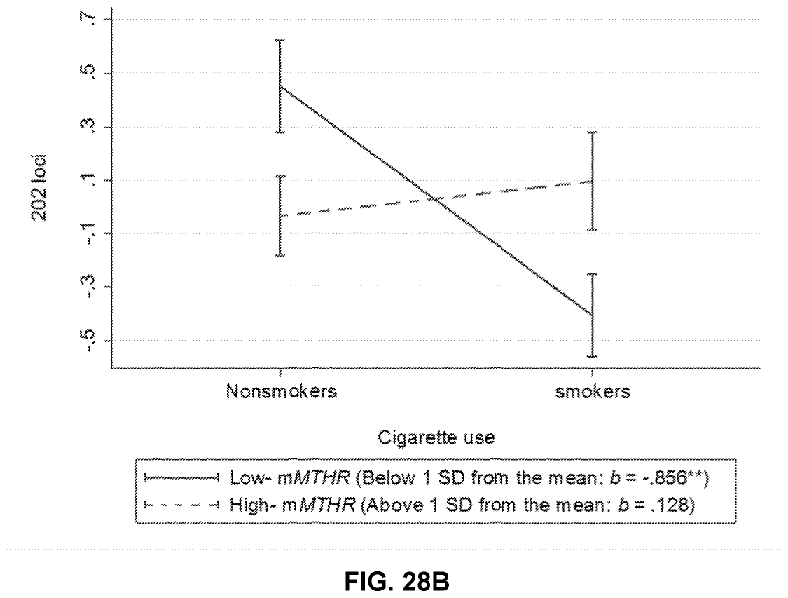

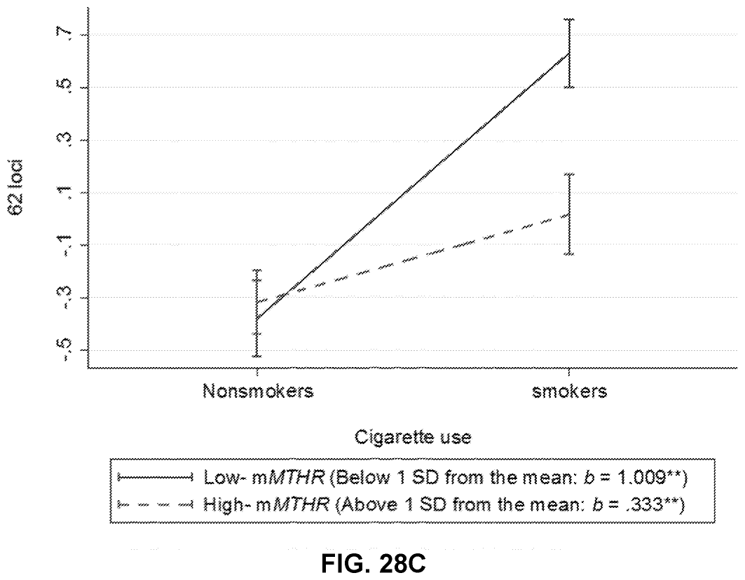

Of the 910 loci identified in the initial report, it was observed that 284 loci showed significant interaction effects involving mMTHFR, suggesting a broad regulatory impact of mMTHFR for the range of toxic exposures inherent in tobacco smoke. In addition, as expected, it was found that the interaction effect for these loci showed a different pattern of regulation by mMTHFR depending on whether the effect of smoking was in the direction of hyper methylation or in the direction of hypo methylation at the specific methylation locus. As can be seen in FIG. 28, which provides the table of results, 89.9% of loci that were hypo methylated in response to smoking demonstrated a negative interaction with mMTHFR. For loci that were hyper methylated in response to smoking 94% demonstrated a positive interaction with mMTHFR. This resulted in a highly significant chi-square Chi-square=188.788, df=1, p=0.000, supporting hypothesized differences in the effect of mMTHFR for hyper vs hypo methylation in response to long-term smoking.

The overall shape of the loci demonstrating a negative interaction with mMTHFR is shown in FIG. 28a. As can be seen, the shape of the interaction effects for those loci showing long-term hypo methylation in response to smoking and that are influenced by variation at mMTHFR shows a cross over interaction effects. The contrast of those 1 sd below the mean on mMTHFR with those 1 sd above the mean on mMTHFR Indicates that there is more exaggerated long-term remodeling in response to smoking among those with lower mMTHFR than for those with higher mMTHFR. As can be seen in FIG. 28b, which illustrates the shape of the interaction effect for those loci showing long-term hyper methylation in response to smoking, and indicates that individuals with higher mMTHFR showed muted hyper methylation in response to smoking whereas those with lower mMTHFR showed more exaggerated hyper methylation.

The observed pattern suggests that variation in mMTHFR has the potential to regulate and predict longer-term methylation remodeling in response to long-term exposure to hyper and hypo methylating agents, and has the potential to predict informative, broad patterns of hyper and hypo methylation that result from such long-term toxic exposure. In particular to cigarette smoke exposure clearly has longer-term effects on an expanding array of CpG sites and the response of these reporters of long-term exposure is, in many cases, moderated by mMTHFR. This will allow development of predictive algorithms utilizing mMTHFR to predict individual differences in methylation profiles in response to smoking, or other hyper or hypo methylating agents, providing long-term risk estimates for particular diseases or conditions that may arise in response to smoking or exposure to other agents.

### REFERENCES FOR EXAMPLE 3

Dogan et al., 2014. The effect of smoking on DNA methylation of peripheral blood mononuclear cells from African American women. BMC Genomics 15:151.

### Example 4: mMTHFR's Role in Regulating Methylation Response to Toxic Exposure from Sources Other than Cigarette Smoke

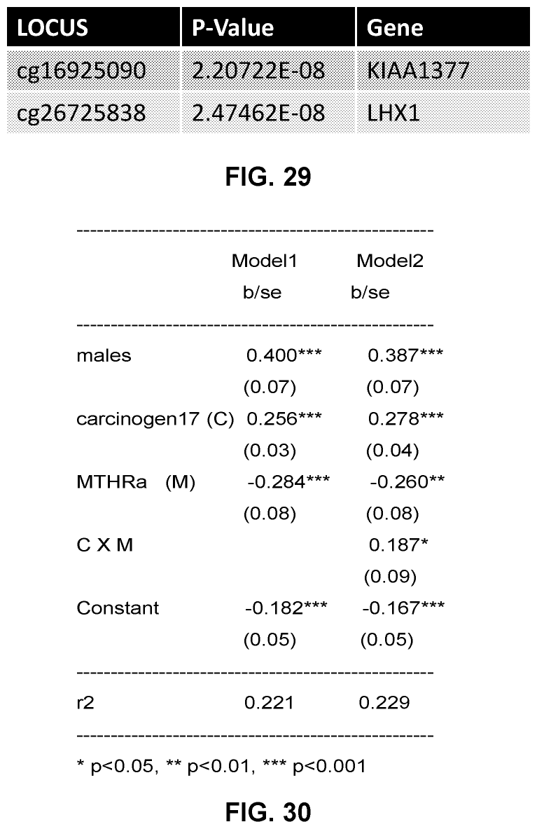

To determine whether mMTHFR might influence response to toxic exposures from sources other than cigarette smoke, we also examined methylation response to toxic exposure to airborne carcinogens. Toxic exposure indices can be generated at the census tract level using the EPA's National-Scale Air Toxic Assessment (NATA). These measures include exposures to individual substances (e.g., heavy metals, such as arsenic and cadmium—also found in tobacco smoke) as well as an aggregate measure of total risk from all carcinogens. We focused on the aggregate measure of total risk from all carcinogens due to airborne toxins, using NATA-linked census tract data, geocoded with participants' addresses. Because NATA includes all hazardous air pollutants (regulated by the Clean Air Act) that are related to cancer (EPA, 2016), we obtained a comprehensive non-smoking index of airborne toxic exposure at each wave for each participant. Using this approach to characterize all participants in the SHAPE sample (sample 2, described above in example 1) we accurately characterized each participant's likely level of exposure to airborne toxins based on the location of their primary residence across a 10-year period. We first examined methylation genomewide, to identify two loci that were significantly associated with NATA specified levels of airborne toxic exposure, even after stringently correctly for total number of genomewide comparisons (See FIG. 29). To examine the role of mMTHFR in regulating the observed hyper methylation response, we then examined whether the association of toxic exposure on these loci was moderated by mMTHFR. As can be seen in FIG. 30, even after controlling for sex, and the main robust main effects of mMTHFR and toxin exposure, the average methylation of the two loci was significantly moderated by mMTHFR in response to differences in level of toxin exposure. Accordingly, there is support for the broader applicability of mMTHFR as a regulator of methylation response to a range of toxic exposures, even when the source of the toxins is not cigarette smoke.

### Example 5: MTHFR Expression Measures Provide an Alternative Reporter of mMTHFR, Yielding Similar Patterns of Moderation of Smoking Effects

Beach and others [2017a] suggested that, because methylene tetrahydrofolate reductase (MTHFR) is the rate-limiting enzyme in the methyl cycle, a pathway critical for folate availability, it is potentially an equivalent and alternative reporter of individual differences in efficiency of methylation repair activities [Stover 2009], and so MTHFR availability should also moderate degree of demethylation at cg05575921 in response to smoking, albeit in the opposite direction to that found for mMTHFR. That is, reduced MTHFR availability, whether indexed via greater mMTHFR or lower gene expression, should enhance demethylation at cg05575921 in response to smoking. Conversely, greater MTHFR availability, whether indexed by lower methylation or greater gene expression, should blunt the impact of smoking on demethylation of cg05575921. Providing initial support for this hypothesis, Beach and others [2017], discussed in example 1, showed that mMTHFR moderated the impact of smoking on CG05575921 demethylation in two independent samples (N=N=293; N=368) of African American young adults. Those with greater mMTHFR showed greater demethylation at cgCG05575921 in response to smoking.

If Beach and other's [2017a] account of MTHFR's role in regulating demethylation at cg05575921 in response to smoking is correct, individual differences in level of MTHFR expression should provide an alternative approach to index MTHFR availability, and so should also moderate smoking's effect on cg05575921. Gene expression, in general, would be expected to show a similar, but reversed, pattern compared to that seen using MTHFR methylation. That is, lower levels of MTHFR expression, should predict greater demethylation at cg05575921 in response to smoking. Conversely, greater MTHFR expression should predict a blunted impact of smoking on demethylation of cg05575921. Accordingly, using a new young adult sample of similar racial composition (African American), sample size (n=334), and sex balance (N=117 males; N=217 females), we conducted a stringent conceptual replication, examining the moderating effect of MTHFR expression on the extent to which self-reported smoking resulted in demethylation at cg05575921. As we did for the prior analyses, we also present analyses including only cotinine verified non-smokers, thereby replicating findings in a subsample that excludes probable under reporters of smoking.

In addition to potential concerns regarding lack of direct examination of MTHFR expression as an index of MTHFR availability, there also are potential concerns regarding lack of controls for the possible impact of secondhand smoke exposure or family history of smoking on methylation in general, and demethylation at cg05575921 in particular. Levels of cotinine are elevated in passive smokers, and so gene expression may also be sensitive to secondhand smoke (Benowitz, et al., 2009), suggesting the value of controlling secondhand smoke effects or other smoking-related family effects that may influence demethylation of cg05575921.

Sample and Measures.

To address the role of MTHFR expression as a moderator of smoking's effect on cg05575921, as well as to add controls for potential secondhand smoke exposure, we examined a sample of young adults from the Family and Community Health Study (FACHs), an ongoing project focused on risk and protective factors for the health of young-adult African Americans who, along with a parent figure, have been followed since childhood. The sample had a mean age of 29 at the time of their blood draw. Details on prior waves of data collection for the FACHs project, general procedures utilized, and sample characteristics are described in prior publications (Beach and others 2017b; Simons and others, in press). Self-reported smoking status at prior waves were used to establish smoking history across an eight year period. Specifically, to assess cigarette smoking across three waves of data collection from ages 21-29 young adults were asked “In the past month, how much did you smoke cigarettes?” Response options included: 0-None at all; 1-Up to one cigarette a day; 2-2 or 3 cigarettes a day; 3-Up to a pack a day; 4—More than a pack a day. Then, scores were summed across waves. To establish robustness of effects, supplemental analyses examined effects using a dichotomous measure of smoking. For the supplemental analyses, anyone who indicated any smoking in the past three months at any of the three assessment waves from age 21 to 29 was classified as a “smoker.” Those who answered “no” at all waves were classified as “non-smokers.”

Blood Based Measures.

A certified phlebotomist drew several tubes of blood at each participant's home, two of which are germane to the current investigation. To examine mRNA we used PAXgene tubes that were spun and then stored in a −80° freezer until use. DNA for the droplet digital PCR used whole blood and was prepared according to previously published protocols (Philibert et al., 2012; Philibert, Beach, Lei, & Brody, 2013). Additional samples of blood were collected for other purposes as well as for storage for future use.

All participants who provided blood at the final wave of assessment at mean age 29.11 (SD=0.74) were eligible for inclusion.

Genomewide mRNA.

To obtain mRNA values, blood samples were collected in a PAXgene tube and frozen at −80 C until use. All available samples were sent to the Rutgers repository. After excluding samples with poor quality (n=81) and samples with no amplification (n=3), we were left with a total sample of N=368 young adults with mRNA, of whom 334 also had all self-report report measures and CG05575921 methylation values included in our analyses. The viable samples were annotated using the Illumina HumanHT-12 v4 BeadChip where 200 ng of total RNA was processed according to the protocol supplied by Illlumina. All samples were randomized prior to array hybridization using either two or three technical replicates. After background subtraction, raw illumina probe data were exported from GenomeStudio software (version 1.1.1). The microarray data set of 47,323 probes was filtered by removing probes with detection threshold of p<0.05, and probes with fewer than three beads present were also excluded, leaving 44,846 probes for analysis. Then, robust Multi-array Average (RMA) normalized data was log 2 transformed after quantile normalization and the quality of the microarray images was inspected visually using the ArrayAnaysis quality control pipeline available online. The results showed that there were no significant batch effects after quantile normalization. Finally, MTHFR gene expression was characterized as the value of the target transcript (ILMN_1734830) located in 1p36.22.

The Illumina HumanHT-12 V4 BeadChip provides coverage for 31,000 annoted genes with more than 47,000 probes derived from the National Center for Biotechnology Information Reference Sequence (RefSeq) release. It has been validated against TaqMan qRT-PCR, and RNA-seq, as well as other microarray platforms and shows good reliability with these alternative assessment methods for high expression transcripts such as MTHFR (Yu, et al., 2015). In the current sample, MTHFR expression was in the 84^{th} percentile of all mRNA transcripts assessed.

Methylation Response at CG05575921.

Methylation Response at CG05575921 was characterized using droplet digital PCR (ddPCR). 1 μg of DNA from each subject was bisulfite converted using an EpiTect Fast 96 DNA Bisulfite kit (Qiagen, Hilden, Germany) according to the manufacturer's direction. The methylation ratio at CG05575921 in each bisulfite-treated sample was then determined using the Smoke Signature™ Assay (IBI Scientific, Peosta, Iowa) and a QX200 Droplet Digital PCR System™ (Bio-Rad, Hercules, Calif.) according to the manufacturer's protocols. Validation of cg05575921 methylation array data using qPCR has been previously published (Dogan et al., 2014) and ddPCR at CG05575921 has been successfully used to quantify reversion of smoking (Philibert et al., 2015). The reliability of the ddPCR method to quantify cg05575921 methylation was also examined by Andersen et al., (2017), showing that this approach is reliably associated with smoking status, as confirmed by serum cotinine. In addition, a direct comparison of Epic methylation array assessment of cg05575921 to ddPCR assessment, along with establishment of a normal range with 400 adult subjects is in preparation (Philibert et al., in preparation), again showing good correspondence (r=0.991) across methods.

Secondhand Smoke Exposure.

Potential concurrent secondhand smoke exposure or other family influences from parents was controlled by using target report of parent's smoking at the time of the blood draw (i.e., did parent(s) use tobacco during the past 12 months?) and secondhand smoke from friends was controlled using report of friend smoking (e.g., during the past 12 months, how many of your closest friends have used cigarettes?) as covariates.

Characterization of Cell Type Variation.

Using a method developed by Cole and others [2011; 2015], we also controlled for individual variation in the relative prevalence of five major cell types. Estimates were derived using relative abundance of gene transcripts encoding canonical markers of monocytes (CD14), natural killer cells (CD16/FCGR3A, CD56/NCAM1), CD4+ and CD8+T-lymphocyte subsets (CD3D, CD3E, CD4, CD8A), and B lymphocytes (CD19).

Analytic Plan.

We used Beta regressions, as we have done previously [Beach and others, 2017a]. Because the dependent variable (degree of methylation in CG05575921) is calculated as a ratio of methylated to unmethylated loci ranging from zero to one, it typically fails the assumption of having a normal distribution [Dolzhenko and Smith 2014]. Beta regressions, utilizing the beta distribution, were introduced by [Ferrari and Cribari-Neto 2004], to handle dependent variables that are bounded in this manner, accommodating the non-normal distribution that is introduced as a consequence.

Power.

Using the G*Power program we examined whether the current sample had sufficient power to reliably detect the main effect of smoking and the moderating effect of MTHFR expression and smoking on CG05575921 with 334 participants. The program indicated a statistical power greater than 80% at α=0.05 for the model used in our regression analysis presented below (effect size of the main effect=0.163; effect size of the interaction effect=0.027).

Results.

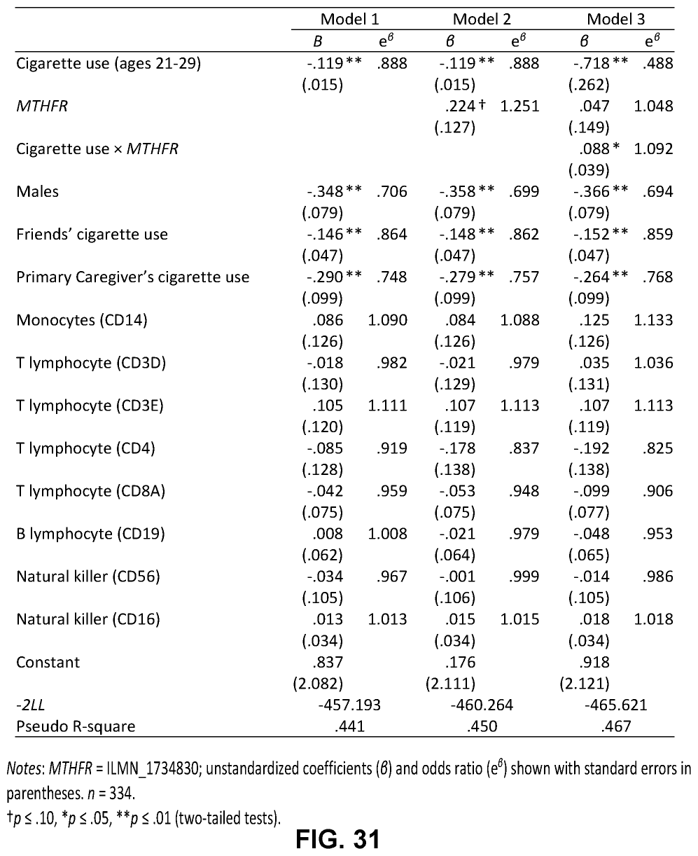

As can be seen in FIG. 31, on step 1 of the beta regression, we entered the main effect of cumulative self-reported smoking across seven years in early adulthood (i.e., waves 5, 6, and 7; ages 21-29). We examined the impact of self-reported smoking in a multivariate context, controlling for the effect of sex, cell-type variation, and effects of secondhand smoke exposure via primary care givers and best friends in all analyses. Replicating prior results, there was a robust effect of smoking which was inversely associated with CG05575921, as were indices of potential secondhand smoke exposure. Indices of cell-type variation were not significantly associated with CG05575921. Further replicating and extending the previously reported finding by Beach et al [2017a], in model 3, it can be seen that MTHFR expression (i.e., level of MTHFR mRNA) moderated smoking's effect on methylation.

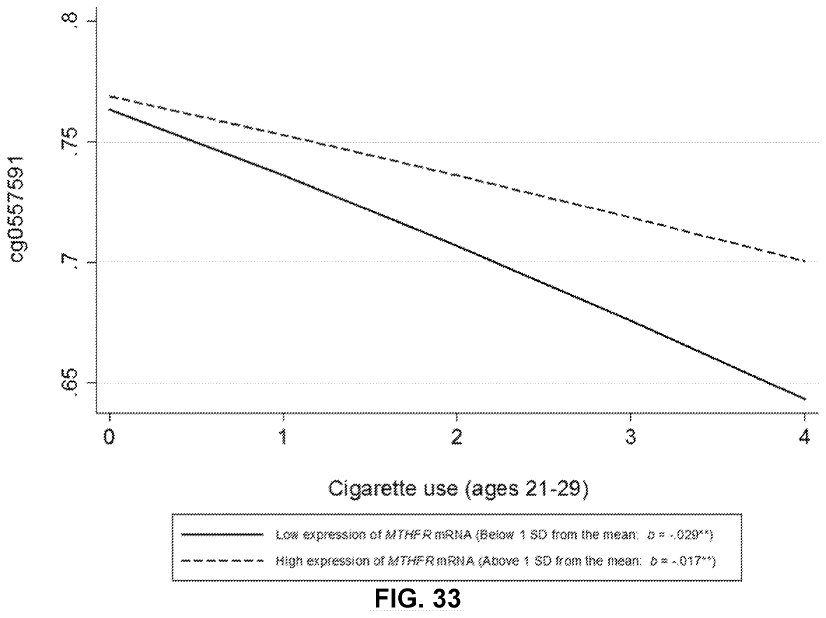

The moderating effect of level of MTHFR expression is explicated in FIG. 33. It can be seen that lower gene expression at MTHFR was associated with a steeper slope relating smoking to CG05575921 whereas greater gene expression was associated with a blunted effect of smoking on CG05575921. That is, the hypothesized pattern of effect was found, reversing that seen previously for mMTHFR.

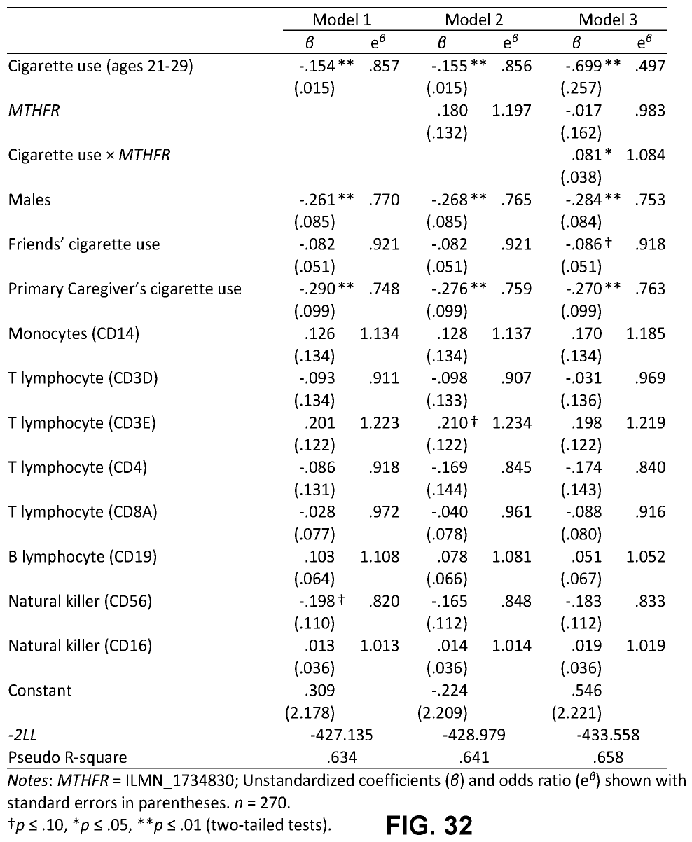

To provide a more stringent test, moderating effects were also examined after excluding those who reported no smoking but had elevated cotinine levels at the time of their blood draw, raising questions regarding the accuracy of their self-reported smoking abstinence. As can be seen in FIG. 32, all significant effects replicated in the cotinine verified subsample, except that secondhand smoking effects via friends was no longer a significant predictor of CG05575921. In addition, the significant interaction effect remained significant and in the same direction as for the full sample.

Conclusions.

Supporting and extending the prior report that MTHFR plays a potentially important regulatory role on demethylation in response to smoking [Beach and others 2017a], we found evidence of regulatory effects of MTHFR using MTHFR expression as an index of MTHFR availability. In addition, observed effects were robust with regard to controls for secondhand smoke exposure as well as cell-type variation. Of central importance, MTHFR expression predicted degree of demethylation in response to smoking, and did so in the predicted direction, with lower gene expression associated with greater impact of smoking on methylation at CG05575921. Results suggest that MTHFR expression may be an alternative method for predicting individual differences in demethylation in response to smoking.

### REFERENCES

Andersen, A M, Philibert, R A, Gibbons, F X, Simons, R L, & Long, J. 2017. Accuracy and utility of an epigenetic biomarker for smoking in populations with varying rates of false self-report. Am J Med Genet Part B. 9999:1-10. DOI: 10.1002/ajmg.b.32555
Beach S R H, Lei M K, Ong M L, Brody G H, Dogan M V, Philibert R A. 2017a. MTHFR methylation moderates the impact of smoking on DNA methylation at AHRR for African American young adults. Am J Med Genet Part B. 174B:608-618. DOI: 10.1002/ajmg.b.32544
Beach, S. R. H., Lei, M. K., Simons, R. L., Barr, A. B., Simons, L. G., Ehrlich, K., Brody, G. H., Philibert, R. A. 2017b. When Inflammation and Depression go Together: The Longitudinal Effects of Parent-Child Relationships. Dev and Psychopathol. 29: 1969-1986. doi:10.1017/S0954579417001523
Benowitz N L, Hukkanen J, Jacob P: 3rd: Nicotine chemistry, metabolism, kinetics and biomarkers. Handb Exp Pharmacol. 2009, 192: 29-60. DOI: 10.10071978-3-540-69248-5_2.
Centers for Disease Control and Prevention. 2008. Smoking-attributable mortality, years of potential life lost, and productivity losses—United States, 2000-2004. MMWR Morb Mortal Wkly Rep 57(45):1226-1228.
Cole, S. W., Levine, M. E., Arevalo, J. M. G., Ma, J., Weir, D. R., Crimmins, E. M. (2015). Loneliness, eudaimonia, and the human conserved transcriptional response to adversity. Psychoneuroendocrinology, 62, 11-17. DOI: 10.1016/j.psyneuen.2015.07.001
Dogan M V, Shields B, Cutrona C, Gao L, Gibbons F X, Simons R, Philibert R A. 2014. The effect of smoking on DNA methylation of peripheral blood mononuclear cells from African American women. BMC Genomics 15:151.
Dolzhenko E, Smith A D. 2014. Using beta-binomial regression for high-precision differential methylation analysis in multifactor whole-genome bisulfite sequencing experiments. BMC Bioinformatics 15(1):1-8.
Ferrari S L, Cribari-Neto F. 2004. Beta Regression for Modelling Rates and Proportions. Journal of Applied Statistics 31 (7):799-815.
Houseman E A, Accomando W P, Koestler D C, Christensen B C, Marsit C J, Nelson H H, Wiencke J K, Kelsey K T. 2012. DNA methylation arrays as surrogate measures of cell mixture distribution. BMC Bioinformatics 13 13:86. DOI: 10.1186/1471-2105-13-86.
Philibert, R. A., Beach, S. R., & Brody, G. H. (2012). Demethylation of the aryl hydrocarbon receptor repressor as a biomarker for nascent smokers. Epigenetics, 7(11), 1331-1338.
Philibert R A, Beach S R H, Lei M-K, Brody G H. 2013. Changes in DNA methylation at the aryl hydrocarbon receptor repressor may be a new biomarker for smoking. Clinical Epigenetics 5(1):19.
Philibert R A, Hollenbeck N, Andersen E, Osborn T, Gerrard M, Gibbons F X, Wang K. 2015. A quantitative epigenetic approach for the assessment of cigarette consumption. Frontiers in Psychology 6:656.
Philibert R A, Dogan M V, Miller S, Noel A, Krukow B, Papworth E. In preparation. The classification and characterization of smoking status by using digital PCR assessments of DNA methylation.
Pidsley R, Wong C C Y, Volta M, Lunnon K, Mill J, Schalkwyk L C. 2013. A data-driven approach to preprocessing Illumina 450K methylation array data. BMC Genomics 14: 293. DOI: 10.1186/1471-2164-14-293
Simons, R. L., Lei, M. K., Beach, S. R. H., Barr, A. B., Simons, L. G., Gibbons, F. X., Philibert, R. A. (in press). Discrimination, Segregation, and Chronic Inflammation: Testing the Weathering Explanation for the Poor Health of Black Americans. Developmental Psychology.
Stover P J. 2009. One-Carbon Metabolism-Genome Interactions in Folate-Associated Pathologies. The Journal of Nutrition 139(12):2402-2405
Yu J, Clifton, P F, Juehne T I, Sinnwell T M, Sawyer C S, Sharma M, Lutz A, Tycksen E, Johnson J R, Minton M R, Klotz E T, Schriefer A E, Yang W, Heinz M E, Crosby S D, Head R D 2015. Multi-platform assessment of transcriptional profiling technologies utilizing a precise probe mapping methodology, BMC Genomics, 16:710.

### Example 6: mMTHFR's Role in Disease and Treatment

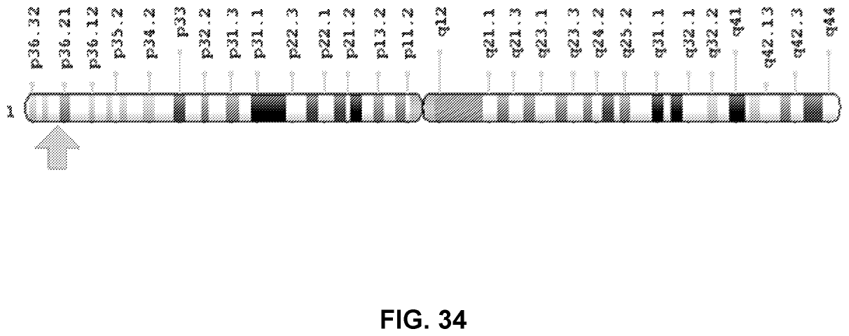

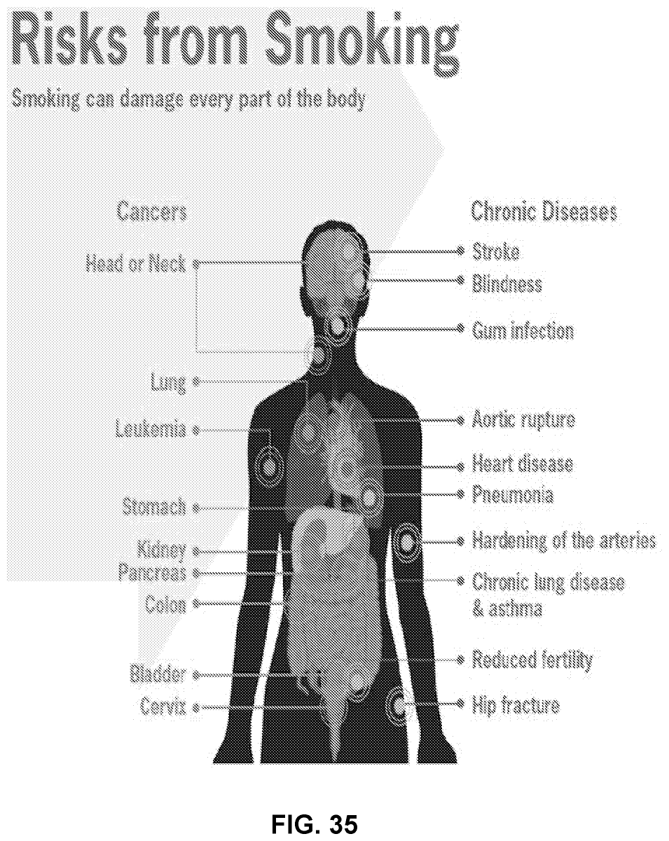

Applicability of mMTHFR to prediction of disease and potentially remediation of disease is suggested by smoking's known connection to a range of disease states. As is portrayed in FIG. 34, taken from the CDC fact sheet on smoking's effects on diseases states (available online) smoking is a leading cause of several disease states. Although not well known at present, patterns of hyper and hypo methylation are likely to have important connections to these diseases that are either general across tissues, specific to particular tissues and organ systems, or specific to diseased tissues. To the extent that mMTHFR exerts a regulatory influence on hyper and hypo methylation responses, it is also likely to have a useful role to play in guiding intervention response to a range of diseases linked to toxin exposure, including use of methylating or demethylating pharmacological agents, or the optimal use of dietary or other supplemental delivery of agents designed to influence methylation. Similarly, for medications, medical conditions, environmental conditions, compounds, enzymes or activity thereof, that have acute or chronic effects on health, have side effects, or influence well-being, due in part or in whole, to changes in methylation, assessment of mMTHFR is likely to play a useful role in guiding use and level of safe or optimal exposure.

Accordingly, application of assessment of mMTHFR in these contexts has the potential to lead to better medical intervention.

1. A method to detect, quantify, or detect and quantify exposure of a subject to methylation modulating agent (MMA) in the subject comprising:
providing a biological sample from the subject;
contacting DNA from the biological sample with bisulfite under alkaline conditions to produce bisulfite-treated DNA;
contacting the bisulfite-treated DNA with a first oligonucleotide probe, wherein the first oligonucleotide probe is complementary to a nucleotide sequence that comprises a CpG dinucleotide in the methylene tetrahydrofolate reductase (MTHFR) gene, wherein the first oligonucleotide probe detects either the unmethylated CpG dinucleotide or the methylated CpG dinucleotide; and
detecting either the unmethylated CpG dinucleotide or the methylated CpG dinucleotide in the MTHFR gene;
wherein methylation of the CpG dinucleotide in the MTHFR gene is associated with amplification of response of the reporter CpG indicating the exposure of the subject to the MMA.

2. The method of claim 1, further comprising the steps of
contacting the bisulfite-treated DNA with a second oligonucleotide probe, wherein the second oligonucleotide probe is complementary to a nucleotide sequence that comprises a CpG dinucleotide within the aryl hydrocarbon receptor repressor (AHRR) gene or other reporter nucleotide sequence specific to the MMA being quantified;
detecting either the unmethylated CpG dinucleotide or the methylated CpG dinucleotide within the AHRR gene or other reporter nucleotide sequence specific to the MMA being quantified; and
conducting a regression analysis using the degree of methylation in the MTHFR gene and the AHRR gene or other reporter nucleotide sequence specific to the MMA being quantified to predict the intensity of the subject's exposure the MMA.

3. The method of claim 1, wherein the CpG dinucleotide in the MTHFR gene is selected from the group consisting of: cg01134491, cg01226883, cg02978542, cg05228408, cg05265975, cg08269394, cg08869383, cg10221637, cg11276438, cg12751404, cg14032528, cg14472778, cg17514528, cg17745097, cg18187189, cg21864959, cg22877851, cg23068701, cg23088157, cg23226134, cg23952195, cg25628740, cg27012203, and any combination thereof.

4. The method of claim 1, wherein the CpG dinucleotide in the MTHFR gene is selected from the group consisting of: cg02978542, cg08269394, cg12751404, cg14032528, cg23068701, cg23226134, cg23952195, and any combination thereof.

5. The method of claim 2, wherein the CpG dinucleotide within the AHRR gene is selected from the group consisting of: cg05575921, cg21161138, cg26703534, and any combination thereof or other reporter nucleotide sequence specific to the MMA being quantified.

6. The method of claim 1, wherein the biological sample is blood or tissue more directly affected by the exposure.

7. The method of claim 6, wherein the biological sample is a mononuclear cell pellet prepared from the biological sample.

8. The method of claim 1, further comprising an amplifying step after the one or more contacting steps.

9. The method of claim 8, further comprising a sequencing step performed after the amplifying step.

10. The method of claim 1, wherein the MMA is selected from the group consisting of: a toxin, a carcinogen, a pharmaceutical compound, a protein, an enzyme, a vitamin, tobacco smoke, a compound found in tobacco smoke, a medical condition, a non-medical condition, and any combination thereof.

11. The method of claim 1, wherein methylation of the CpG dinucleotide in the MTHFR gene is associated with amplification of response of the reporter CpG and indicates the level of cumulative exposure to the MMA.

12. The method of claim 11, wherein the reporter CpG nucleotide is specific to the MMA.

13. The method of claim 1, further comprising the step of determining the subject's actual exposure to MMA or determining the subject's predicted exposure to the MMA and wherein methylation of the CpG dinucleotide in the MTHFR gene is associated with amplification of response of the reporter CpG indicating the exposure of the subject to the MMA.

14-17. (canceled)

18. The method of claim 1, further comprising the step of treating the subject for MMA exposure.

19. The method of claim 18, wherein the step of treating the subject for MMA exposure comprises administering a pharmaceutical to the subject.

20. The method of claim 18, wherein the step of treating the subject for MMA exposure includes administering behavioral therapy, psychiatric therapy, psychotherapy, a pharmaceutical or a combination thereof to the subject.

21. A kit configured to determine the methylation status of at least one CpG dinucleotide, the kit comprising:
at least one first oligonucleotide probe, wherein the first oligonucleotide probe is complementary to a nucleotide sequence that comprises a CpG dinucleotide in the methylene tetrahydrofolate reductase (MTHFR) gene, wherein the first oligonucleotide probe detects either the unmethylated CpG dinucleotide or the methylated CpG dinucleotide.

22-29. (canceled)


SEQUENCE LISTING (4 sequences)

SEQ ID NO: 1 - DNA, 1120 bp, Artificial Sequence
  Region of MTHFR gene
cgtgaagcagggtagacgcttcgagagccctggctgcggtccccaggccccacccgctgccacctgcgggcccagattggcccggccccacccccggcaacgcctctctcagtcccttagcaaccgccccctccccaggccgactccgccggcttcttaccagctcctcgggggtgcgggtctcacgctcaccgcagcagcagcaccacctgcagcagcagcacagagaccccctgcaccccgccatcttcctcctttactgccactctggacccctctaccaaccccctcccagccaggatctgcgcctcacgtgactggccccgggacggtcacgtggccctctcgagctctgggactgagaccaggagtggctgcagacgggtggggcgaggactcgcgtcacatgacgataaaggcacggcctccaacgagacctgtgggcacggccatgttgggggcggggcttccggtcacccgcgccggtggtttccgccctgtaggcccgcctctccagcaacctgacacctgcgccgcgccccttcactgcgttccccgcccctgcagcggccacagtggtgcggccggcggccgagcgttctgagtcacccgggactggagggtgagtgacggcgaggccggggtcgccgggagggagatcctggagccggcaaacaacctcccgggggcaaggacgtgcttgtgggcggggagcgctggaggccggcctgcctctcttcttggggggggctgccgcctcccttgcgcacccttcgcgggattagtgtaactcccaatggctaccacttccagcgaccgccaaccctcaagcgaagactgactttggctccctgcctggacggaggggcccctgagccaggggtgacgatcccgcccctctgaccggcccaggcccgtgtcctcgcccccatcggtgactcagtgacctggtgactggattctcggccacctgggcgccgagacggcttccggctcctgccttttaaacctgcctccccggcgatcacctggagaagagcgctgggcccggggcactgcggtccctggcgcccactgcgtcccgctgcgcacgggggtccgccgggacctttctgggagt

SEQ ID NO: 2 - DNA, 1122 bp, Artificial Sequence
  region of AHRR gene on chromosome 5
aattggcttcaggggactgggagcaatgtggcttcgtttctgggcatcacggcatcacccagacgcacagctggacctgccttctggctcctcctccaccagggactgtgggtcagaccgagtcacaggcccaggtgctcgtggtgggggccagatggggtggccttgggttcccggtgacaccttgctctgagtctcctctgtgtgaagaaacagagggcccttagcaggaccccatgggaagcctgagcccccaagggagccactctgctccagcccttgctgtgggcacagggtcgcccgaggccttggtgctgacttcacattctcgctgttccaaatgagccccttcttggttgcagtgaactctgcagccgtgggcggacggctggtaatggccctgagactctccaaggtggctgaggttctgcgcggttggcagagtgctggcaggacacaggggttgcccaggctacagatccgggttcaggcccagccgtggggactgttcacctctgagagggcagccctgccccgcggccctggccctgccctgcacccggctgggtctcatctgacacgcagccttccagcctctcattgcccgaggggtgggccctgggagtggtcctggcagggcccctctctgcagaccctgcgggaccagcaggccgggcggtggctgggaggcaggggacaggttggcctctgcggtcttggaggcacagagctcctcccgccctgggcctgtggccccctcctgctggcccagctgtgccttgtaagggccacggtgatggcccacctttctcaccaatgcccaggaaggacaccgcgctcccagctcccttcctccccatccactcaccatgggcaactgcggtggctgggcggagggggggtctctctccacatcacagaggaggagactggggctggagaggtgggggatgggggctccctcccggggcggaccctcaggtgggacccccaggctcagggagggcggggggacctggggaggccgtggccgggtccgcccgccccgtgtgcacccggagcgcccgcgcggaccccgagccccattccccgccacgcgcctggcgctccccgcgctcctgg

SEQ ID NO: 3 - DNA, 307 bp, Artificial Sequence
  target region for cg14032528
gagctcgagagggccacgtgaccgtcccggggccagtcacgtgaggcgcagatcctggctgggagggggttggtagaggggtccagagtggcagtaaaggaggaagatggcggggtgcagggggtctctgtgctgctgctgcaggtggtgctgctgctgcggtgagcgtgagacccgcacccccgaggagctggtaagaagccggcggagtcggcctggggagggggcggttgctaagggactgagagaggcgttgccgggggtggggccgggccaatctgggcccgcaggtggcagcgggtggggc

SEQ ID NO: 4 - DNA, 307 bp, Artificial Sequence
  bisulfite converted target region for cg14032528.
  misc_feature: N in SEQ ID NO. 4 indicates bisulfite converted nucleotide
  misc_feature (6)..(6): n is a, c, g, or t
  misc_feature (17)..(17): n is a, c, g, or t
  misc_feature (23)..(23): n is a, c, g, or t
  misc_feature (28)..(28): n is a, c, g, or t
  misc_feature (40)..(40): n is a, c, g, or t
  misc_feature (47)..(47): n is a, c, g, or t
  misc_feature (111)..(111): n is a, c, g, or t
  misc_feature (160)..(160): n is a, c, g, or t
  misc_feature (167)..(167): n is a, c, g, or t
  misc_feature (176)..(176): n is a, c, g, or t
  misc_feature (184)..(184): n is a, c, g, or t
  misc_feature (203)..(203): n is a, c, g, or t
  misc_feature (206)..(206): n is a, c, g, or t
  misc_feature (212)..(212): n is a, c, g, or t
  misc_feature (228)..(228): n is a, c, g, or t
  misc_feature (252)..(252): n is a, c, g, or t
  misc_feature (258)..(258): n is a, c, g, or t
  misc_feature (270)..(270): n is a, c, g, or t
  misc_feature (286)..(286): n is a, c, g, or t
  misc_feature (298)..(298): n is a, c, g, or t
gagttngagagggttangtgatngtttnggggttagttangtgaggngtagattttggttgggagggggttggtagaggggtttagagtggtagtaaaggaggaagatggnggggtgtagggggtttttgtgttgttgttgtaggtggtgttgttgttgnggtgagngtgagattngtattttngaggagttggtaagaagtnggnggagtnggtttggggagggggnggttgttaagggattgagagaggngttgtngggggtggggtngggttaatttgggttngtaggtggtagngggtggggt